SEQUENCE LISTING (7 sequences)

SEQ ID NO: 1 - DNA, 21 bp, synthetic construct
  source 1..21: mol_type = other DNA
gaacatgatcgatccatgtca

SEQ ID NO: 2 - RNA, 19 nt, synthetic construct
  source 1..19: mol_type = other RNA
catccataaagaaggcata

SEQ ID NO: 3 - RNA, 19 nt, synthetic construct
  source 1..19: mol_type = other RNA
ccacttattggccctcatt

SEQ ID NO: 4 - RNA, 19 nt, synthetic construct
  source 1..19: mol_type = other RNA
gacttgttctgtcgcttct

SEQ ID NO: 5 - RNA, 48 nt, synthetic construct
  source 1..48: mol_type = other RNA
ggccgtgatggtcagataattggatccaattatctgaccatcacggcc

SEQ ID NO: 6 - RNA, 21 nt, synthetic construct
  source 1..21: mol_type = other RNA
tacagaggcaagcggtgaact

SEQ ID NO: 7 - RNA, 22 nt, synthetic construct
  source 1..22: mol_type = other RNA
gtgtaacacgtctatacgccca

(19) United States
(12) Patent Application Publication
(10) Pub. No.: US 2025/0297323 A1
(43) Pub. Date: Sep. 25, 2025

(54) METHODS OF USING LONG NON-CODING RNA-8 (TROLL-8) AS A TARGET FOR CANCER DETECTION AND TREATMENT

(71) Applicant: H. LEE MOFFITT CANCER CENTER AND RESEARCH INSTITUTE, INC., Tampa, FL (US)

(72) Inventors: Elsa FLORES, Tampa, FL (US); Marco NAPOLI, Tampa, FL (US)

(21) Appl. No.: 18/864,311

(22) PCT Filed: May 10, 2023
(86) PCT No.: PCT/US2023/066815

§ 371 Date: Nov. 8, 2024

Related U.S. Application Data
(60) Provisional application No. 63/340,311, filed on May 10, 2022.

(51) Int. Cl. C12Q 1/6886; A61K 38/45; C12N 15/113

(52) U.S. Cl. CPC C12Q 1/6886; A61K 38/45; C12N 15/113; C12Q 2600/112; C12Q 2600/118; C12Q 2600/158; C12Y 203/01021

(57) ABSTRACT

Disclosed are long non-coding RNAs (IncRNAS) for TROLL-8. It is shown herein that IncRNAs TROLL-8, is a suitable target for cancer therapies and can be used to make prognostic determinations about a cancer. Specifically, the disclosure provides a method of assessing tumor grade and/or progression of a cancer and/or metastasis in a subject comprising obtaining a tissue sample from a subject and measuring the expression level of the long non-coding RNA for TROLL-8; wherein the higher the level of IncRNA for TROLL-8, the greater the severity and/or invasiveness of the tumor is indicated. Further disclosed is a method of assessing the efficacy of a cancer treatment regimen administered to a subject, the method comprising measuring the expression level of the long non-coding RNA for TROLL-8 in a tissue sample from the subject relative to a control.

### II. CROSS REFERENCE TO RELATED APPLICATIONS

This application claims the benefit of U.S. Provisional Application No. 63/340,311, filed on May 10, 2022, which is incorporated herein by reference in its entirety.

### I. GOVERNMENT SUPPORT

This invention was made with government support under Grant No. R35CA197452 awarded by the National Institutes of Health. The government has certain rights in the invention.

### REFERENCE TO SEQUENCE LISTING

The sequence listing submitted on May 10, 2023, as an .XML file entitled “10110-402WO2.XML” created on May 10, 2023, and having a file size of 7,098 bytes is hereby incorporated by reference pursuant to 37 C.F.R. § 1.52(e)(5).

### III. BACKGROUND

Cancer metastasis is the leading cause of death in cancer patients. Multiple pathways have been found to increase cancer progression and metastasis including the activation of the PI3K/AKT pathway and the gain-of-function mutation of the tumor suppressor TP53, which are the two most frequent driving mutations in a broad variety of human cancers. Therefore, investigating the mechanistic interplay between these pathways is of the utmost importance for the identification of novel therapeutic opportunities against the progression of metastatic cancers.

### IV. SUMMARY

Disclosed are methods and compositions related to long non-coding RNAs (lncRNAs) for TROLL-8 in the detection and treatment of breast cancer.

In one aspect, disclosed herein are methods of assessing tumor grade and/or progression of a cancer and/or metastasis (such as, for example, breast cancer, including, but not limited to triple negative breast cancer (TNBC) or invasive ductal carcinoma (IDC)) in a subject comprising obtaining a tissue sample from a subject and measuring the expression level of the long non-coding RNA for TROLL-8; wherein the higher the level of lncRNA for TROLL-8, the greater the severity and/or invasiveness of the tumor is indicated. In some aspects, the cancer comprises a cancer with a KRAS^{G12C} mutation or p53 mutation.

Also disclosed herein are methods of assessing the efficacy of a cancer treatment regimen administered to a subject comprising obtaining a tissue sample from a subject and measuring the expression level of the long non-coding RNA for TROLL-8 relative to a control.

In one aspect, disclosed herein are methods of assessing the efficacy of a cancer treatment of any preceding aspect, wherein when the expression level of lncRNA for TROLL-8 is i) higher than a negative control, ii) equivalent to or has not decreased relative to a positive control and/or equivalent to or has not decreased relative to a positive control; indicates that the treatment regimen is not efficacious. In one aspect, disclosed herein are methods of assessing the efficacy of a cancer treatment wherein the positive control is a reference gene or pretreatment sample from the subject whose cancer treatment regimen is being assessed.

Also disclosed herein are methods of detecting the presence of a cancer (such as, for example, breast cancer, including, but not limited to triple negative breast cancer (TNBC) or invasive ductal carcinoma (IDC)) in a subject comprising obtaining a tissue sample from the subject and assaying the tissue sample for the presence and/or expression level of the long non-coding RNA for TROLL-8; wherein the presence or an increase in lncRNA for TROLL-8, indicates the presence of a cancer in the tissue sample from the subject. In some aspects, the cancer comprises a cancer with a KRAS^{G12C} mutation or p53 mutation.

In one aspect, disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) in a subject comprising obtaining a tissue sample from a subject receiving a cancer treatment regimen and measuring the expression level of the long non-coding RNA for TROLL-8; wherein when the expression level of lncRNA for TROLL-8 is i) higher than a negative control and/or equivalent to or has not decreased relative to a positive control; indicates that the treatment regimen is not efficacious; and wherein the method further comprises changing the treatment regimen when the treatment regimen is not efficacious. In some aspects, the cancer comprises a cancer with a KRAS^{G12C} mutation or p53 mutation.

Also disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) in a subject comprising i) obtaining a tissue sample from the subject; ii) assaying the tissue sample for the presence and/or expression level of the long non-coding RNA for TROLL-8; wherein the presence of lncRNA for TROLL-8 indicates the presence of a cancer in the tissue sample from the subject; and iii) administering to a subject an agent that knocks down expression of TROLL-8 or increases expression of carnitine palmitoyltransferase 1A (CPT1A). In some aspects, the cancer comprises a cancer with a KRAS^{G12C} mutation or p53 mutation.

In one aspect, disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) of any preceding aspect, wherein expression of TROLL-8 is knocked down through the administration of one or more RNA-targeted therapeutics including, but not limited to antisense oligonucleotides, siRNA (such as, for example, SEQ ID NO: 2, SEQ ID NO: 3, and/or SEQ ID NO: 4), shRNA, ribozymes, transcription activator-like effector nucleases (TALEN), zinc finger nucleases (ZFNs) and/or clustered regularly interspaced short palindromic repeats/associated (CRISPR/Cas) nucleases.

Also disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) of any preceding aspect, wherein the treatment comprises administering to the subject carnitine palmitoyltransferase 1A (CPT1A) or a vector that overexpresses CPT1A.

In one aspect, disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) of any preceding aspect, further comprising the administration of a second anti-cancer agent and/or immunotherapy.

### V. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, and 1F show that TAp63 regulated oncogenic lncRNA-8 (TROLL-8) expression positively correlates with human breast cancer progression. Pan-cancer analysis of TROLL-8 expression in the TCGA database in breast cancer molecular subtypes (1A). Data were analyzed with one-way ANOVA. Pairwise comparisons with significant P-values are demonstrated. Kaplan-Meier curves showing overall breast cancer survival data based on TROLL-8 expression in the TCGA database in BRCA patients (1B), TNBC patients (1C), and IDC patients (1D). IDC=invasive ductal carcinoma. Quantification of the ISH scores of TROLL-8 expression in the indicated TMA. Data were analyzed with one-way ANOVA (1E) and two-tailed Student's t test (1F). Asterisk vs. normal breast tissue (NB), P<0.05. The TMA whisker boxplots represent the individual data points and median.

FIGS. 2A, 2B, 2C, 2D, 2E, and 2F show that TROLL-8 interacts with proteins enriched in cellular metabolism. FIG. 2A shows a schematic and Venn diagram showing the RNA-protein interaction microarray and the Ingenuity Pathway Analysis (IPA) of the proteins interacting with TROLL-8. FIG. 2B shows enriched canonical pathways of the proteins interacting with TROLL-8 defined by IPA. Agilent Seahorse XF Cell Mito Stress Test Profile showing oxygen consumption rate (OCR) kinetics with key parameters of mitochondrial function (2C). Key parameters of mitochondrial function were compared before and after TROLL-8 knockdown (2D). OCR in control (siNT, blue bar) cells in basal respiration and ATP production was compared with that in TROLL-8 knockdown (siTROLL-8, red bar) cells. OCR was measured by the Agilent Seahorse XF mitochondrial fuel oxidation stress test to compare the glucose, palmitate and glutamine demands in siNT and siTROLL-8 cells supplemented with the corresponding fuels (2E). OCR measurements of mitochondrial dependency and flexibility for each fuel source upon addition of inhibitor of the corresponding fuel source (2F). OCR values were normalized to siNT without treatment for each group. A two-tailed Student's t test was used, and results with P<0.05 were considered as statistically significant. Abbreviations: FCCP, carbonyl cyanide-p-trifluoromethoxyphenyl hydrazone; Rot/AA, rotenone and antimycin A; ns, not significant.

FIGS. 3A, 3B, 3C, 3D, 3E, 3F, and 3G show that TROLL-8 downregulation leads to compromised fatty acid oxidation (FAO) and long-chain fatty acids (LCFAs) accumulation. FIG. 3A shows a volcano plot comparing the metabolite accumulation patterns from control (siNT) and TROLL-8 knockdown (siTROLL-8) groups obtained from LC-MS targeted metabolomics experiments; visualization using GraphPad Prism. The plot represents all available metabolites (298 metabolites). Individual metabolites are plotted according to their fold change and significance. Significance is indicated by either red dots (downregulation in siTROLL-8 group) or green dots (upregulation in siTROLL-8 group) on the plot with P<0.05 were considered as statistically significant. FIG. 3B shows a table showing metabolites with significant upregulated levels after TROLL-8 knockdown. FIG. 3C shows a brief schematic demonstrates the U-¹³C-palmitate tracing workflow to track the accumulation of input U-¹³C-Palmitate, the generation of FAO product ¹³C-citrate, and the de novo synthesized palmitate. Labeling patterns following isotope tracing with U-¹³C-palmitate of cellular citrate (3D), U-¹³C-palmitate (3E) and labeling patterns following isotope with U-¹³C-glutamine (3F), and U-¹³C-glucose (3G) in MCFCA1D cells expressing siNT or siTROLL-8. For all the metabolites, partially labeled intermediates, and fully labeled isotopomers are shown. Comparisons between siNT and siTROLL-8 groups with P<0.05 were considered statistically significant. A two-tailed Student's t test was used.

FIGS. 4A, 4B, 4C, 4D, and 4E shows that TROLL-8 interacts with the FAO rate-limiting enzyme, CPT1A. FIG. 4A shows a schematic of cytosolic fatty acid oxidation in the mitochondria. FIG. 4B shows a representative immuno blot analysis for the CPT1A protein pulled down by the indicated in vitro transcribed and biotin-labeled lncRNAs. n=3 biological replicates. Targeted LC-MS metabolomics revealed L-carnitine (4C) and available long chain fatty acyl carnitine (4D) in MCFCA1D cells expressing siNT or siTROLL-8 and treated with vehicle or 10 μM Etomoxir. Comparisons between siNT and siTROLL-8 groups with P<0.05 were considered statistically significant. A two-tailed Student's t test was used. FIG. 4E shows a representative immuno-blot of CPT1A in cellular fractions, cytosol or mitochondria, of MCFCA1D cells expressing siTROLL-8 of CPT1A. n=3 biological replicates.

FIGS. 5A, 5B, 5C, 5D, 5E, 5F, 5G, and 5H show that TROLL-8 regulated CPT1A post-translational modifications mediate its activity. Quantification of cellular acetyl-CoA was performed by LC-MS analysis in MCFCA1D cells expressing siNT (dots) and siTROLL-8 (square) (5A). The relative changes of global protein acetylation are represented by immuno blot in MCFCA1D cells expressing siNT siNT and siTROLL-8 (5B). Cellular extracts from MCFCA1D cells expressing siNT and siTROLL-8 were resolved by SDS-PAGE and Coomassie blue staining. The whole bands demonstrated by immuno blot in (5B) were retrieved and analyzed by mass spectrometry (MS). Bar plots displayed 20 MS detected metabolic proteins with changed acetylation status in MCFCA1D cells expressing siNT (5C) and siTROLL-8 (5D). The raw intensity is converted into a relative abundance by dividing the intensity of modified or unmodified peptides by the sum of all peptides (both modified and unmodified). Bar graph showing relative abundance of acetylated and unmodified peptides within each of the 20 metabolic proteins. Quantification of the ratio of the relative abundance of acetylated peptides in MCFCA1D cells expressing siTROLL-8 compared to MCFCA1D cells expressing siNT for each of the 20 metabolic proteins shown as bar graph (5E) and the peptide acetylation modified sequence in CPT1A is shown in a table (5F). Volcano plot of LC-MS detected CPT1A interacting proteins with or without significant affinity change for CPT1A in MCFCA1D cells expressing siTROLL-8 (5G). Three independent immunoprecipitations were performed for sample collection. A two-tailed Student's t test was used and P<0.05 were considered as statistically significant. Significant affinity fold change >1.5 was highlighted in red. The only acetyltransferase, ACAT1 was revealed in the volcano plot. Ingenuity Pathway Analysis (IPA) demonstrated ACAT1 involved metabolic pathways (5H).

FIGS. 6A, 6B, 6C, 6D, 6E, 6F, 6G, and 6H show that expression of CPT1A and its hypo-acetylated form restore TROLL-8 knockdown to induce mitochondrial respiration and tumorigenesis in breast cancer cells. Representative immuno-blot demonstrated endogenous interaction between ACAT1 and CPT1A. Whole cell lysates from CA1D cells were prepared and immunoprecipitations (IPs) were performed with antibodies against the indicated proteins. Immunocomplexes were then blotted with the antibody against CPT1A (6A). Representative immuno-blot displayed enhanced interaction between ACAT1 and CPT1A after TROLL-8 silencing in MCFCA1D cells. IPs of whole cell lysates from MCFCA1D expressing siNT or siTROLL-8 were performed with indicated antibodies and immunocomplexes were immunoblotted with a CPT1A antibody (6B). Targeted LC-MS—comparison of relative pool size (%) of free cellular carnitine (6C) and fatty acyl-carnitine in MCFCA1D cells expressing wild type (WT) CPT1A or CPT1A acetylation mutants (K148Q or K148R) (6D). Quantification and comparison of key parameters of mitochondrial function after TROLL-8 silencing and/or CPT1A overexpression were analyzed by Agilent Seahorse XF Cell Mito Stress Test (6E & 6F). Bar plots showed the OCR comparison among siNT, siTROLL-8 and/or CPT1A overexpression groups in basal respiration (6E) and ATP production (6F). Quantification and bright field representative images of anchorage-independent colony formation of MCFCA1D cell line in soft agar assay (per 10× field). Data are n=3 biological replicates. Asterisks indicate statistical significance with p<0.05, two-tailed t test (6H).

FIG. 7 shows a schematic of the underlying mechanism of action of TROLL-8. When TROLL-8 is silenced, e.g., the tumor metastasis suppressor and cellular metabolism regulator TAp63 inhibits the expression of TROLL-8, TROLL-8 regulates mitochondrial fuel oxidation, especially FAO, which is indicated by reduced fatty acyl-carnitine and citrate and leads to accumulation of LCFAs. TROLL-8 interacts with proteins enriched in cellular metabolism and regulates cellular availability of acetyl-CoA, and global protein acetylation. TROLL-8 interacts with FAO rate-limiting enzyme, CPT1A, to regulate its activity, acetylation status, and interaction with the acetyltransferase ACAT1.

FIG. 8 shows TROLL-8 expression increases as human breast cancer progression in MCF10 progression model. Quantification of the RNA levels of TROLL-8 in MCF10 progression model, including MCF10A, DCIS, and CA1D cells. Asterisks indicate significant difference with p<0.05, two-tailed t test.

FIG. 9 shows TROLL-8 interacting metabolic protein candidates can be categorized into five major classes. Ingenuity Pathway Analysis (IPA) showed the canonical pathways of the metabolic proteins that can be categorized into fatty acid metabolism, amino acid biosynthesis, purine metabolism, NAD metabolism, cellular metabolic signaling pathway classes.

FIGS. 10A, 10B, 10C, 10D, 10E, 10F, 10G, and 10H show TROLL-8 knockdown leads to compromised glutaminolysis and LCFA accumulation in breast cancer cell lines. Labeling patterns of TCA cycle intermediates involved in glutamine metabolism following isotope tracing with U-¹³C-glutamine of glutaminolysis, alpha-ketoglutaric acid (10A), succinic acid (10B), fumarate (10C), and malate (10D), cellular petroselinic acid/elaidic acid/oleate (10E), labeling pattern of cellular petroselinic acid/elaidic acid/oleate following isotope tracing with U-¹³C-glucose (10F) and U-¹³C-palmitate (10G) in MCFCA1D cells expressing siNT or siTROLL-8. For all the metabolites, partially labeled intermediates, and fully labeled isotopomers are shown. Comparisons between siNT and siTROLL-8 groups with P<0.05 were considered as statistically significant. A two-tailed Student's t test was used. A brief schematic demonstrates the U-¹³C-glutamine and U-¹³C-glucose tracing workflow to track the accumulation of glutaminolysis intermediates and de novo synthesized LCFAs (10H).

FIGS. 11A, 11B, 11C, and 11D show TROLL-8 silencing did not change CPT1A expression or its activity on short chain or medium chain fatty acid. Quantification of the RNA levels of TROLL-8 (11A) and CPT1A (11B) in MCFCA1D cells expressing siNT and siTROLL-8 (11A and 11B). Targeted LC-MS metabolomics of short chain fatty acyl carnitine levels (11C) and medium chain fatty acyl carnitine levels (11D) in MCFCA1D cells expressing siNT and siTROLL-8. A two-tailed Student's t test was used and P<0.05 were considered as statistically significant.

FIG. 12 shows TROLL-8 silencing induces significant affinity change between CPT1A and metabolic proteins. Ingenuity Pathway Analysis (IPA) showed the canonical pathways of the proteins with significant higher or lower affinity change (fold change >1.5) for CPT1A when comparing MCFCA1D cells expressing siTROLL-8 to those expressing siNT.

FIG. 13 shows TROLL-8 silencing enhances the interaction between CPT1A and the acetyltransferase ACAT1 in another TNBC cell line, MCFDCIS. Representative immuno-blot displayed enhanced interaction between ACAT1 and CPT1A after TROLL-8 silencing in MCFDCIS cells. IPs of whole cell lysates from MCFDCIS cells expressing siNT or siTROLL-8 DCIS s were performed with indicated antibodies and immunocomplexes were immunoblotted with CPT1A antibody.

### VI. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHOD OF TREATING CANCER

Long non-coding RNAs (lncRNAs) are regulatory RNAs with no or little protein-coding potential. They function as additional regulators of gene transcription either in cis or trans based on their sequence matching or secondary/tertiary structures. They also serve as decoys, scaffolds, or guides to maintain the spatial-temporal architecture of transcriptional and translational programs on either gene expression or cellular events, including cancer metastasis and metabolism.

Advanced breast cancer metastasis is the major cause of relapse and death in women. However, no effective treatment exists for the metastatic stage of breast cancer. TAp63, one member of the p53 family, is a tumor suppressor in breast cancer metastasis and regulates lipid and glucose metabolism. RNA-seq analysis identified its lncRNA targets, which also differentially expressed during breast cancer progression using MCF10 model. Among them, expression of the oncogenic lncRNA TROLL-8 is significantly higher in triple negative breast cancer (TNBC) molecular subtypes and is negatively correlated with TNBC patient overall survival rate. TROLL-8 interacts with proteins that are enriched in metabolic pathways, detected by protein microarray and Ingenuity Pathway Analysis (IPA). Specifically, seahorse assays demonstrated that TROLL-8 increases breast cancer oxidation pathways. Silencing of TROLL-8 leads to compromised fatty acid oxidation (FAO), which contributes to accumulated long-chain fatty acids (LCFAs) in the breast cancer cells. The rate-limiting enzyme of FAO, carnitine palmitoyltransferase 1A (CPT1A) interacts with TROLL-8, and we show herein that CPT1A contributes to TROLL-8 silencing impaired breast cancer migration. TROLL-8 regulates CPT1A activity and acetylation through blocking its physical interaction with the acetyltransferase ACAT1.

Our study emphasized the potential functionalities of the oncogenic lncRNA TROLL-8 in breast cancer metastasis and metabolism through regulating the FAO rate-limiting enzyme CPT1A activity and post-translational modification. Abnormal expression of TROLL-8 can thus be adopted as diagnostic/prognostic biomarkers, or therapeutic targets for breast cancer control and management.

In one aspect, disclosed herein are methods of assessing tumor grade and/or progression of a cancer and/or metastasis (such as, for example, breast cancer, including, but not limited to triple negative breast cancer (TNBC) or invasive ductal carcinoma (IDC)) in a subject comprising obtaining a tissue sample from a subject and measuring the expression level of the long non-coding RNA for TROLL-8; wherein the higher the level of lncRNA for TROLL-8, the greater the severity and/or invasiveness of the tumor is indicated. In some aspects, the cancer comprises a cancer with a KRAS^{G12C} mutation or p53 mutation.

Also disclosed herein are methods of assessing the efficacy of a cancer treatment regimen administered to a subject comprising obtaining a tissue sample from a subject and measuring the expression level of the long non-coding RNA for TROLL-8 relative to a control.

In one aspect, disclosed herein are methods of assessing the efficacy of a cancer treatment, wherein when the expression level of lncRNA for TROLL-8 is i) higher than a negative control, ii) equivalent to or has not decreased relative to a positive control and/or equivalent to or has not decreased relative to a positive control; indicates that the treatment regimen is not efficacious. In one aspect, disclosed herein are methods of assessing the efficacy of a cancer treatment wherein the positive control is a reference gene or pretreatment sample from the subject whose cancer treatment regimen is being assessed.

Also disclosed herein are methods of detecting the presence of a cancer (such as, for example, breast cancer, including, but not limited to triple negative breast cancer (TNBC) or invasive ductal carcinoma (IDC)) in a subject comprising obtaining a tissue sample from the subject and assaying the tissue sample for the presence and/or expression level of the long non-coding RNA for TROLL-8; wherein the presence or an increase in lncRNA for TROLL-8, indicates the presence of a cancer in the tissue sample from the subject. In some aspects, the cancer comprises a cancer with a KRAS^{G12C} mutation or p53 mutation.

The disclosed compositions can be used to treat any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer, brain cancer; nervous system cancer, head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer, skin cancer, hepatic cancer; melanoma; squamous cell carcinomas of the mouth, throat, larynx, and lung; cervical cancer; cervical carcinoma; breast cancer (including, but not limited to triple negative breast cancer (TNBC) or invasive ductal carcinoma (IDC)); genitourinary cancer, pulmonary cancer, esophageal carcinoma; head and neck carcinoma; large bowel cancer, hematopoietic cancers; testicular cancer; and colon and rectal cancers.

Accordingly, in one aspect, disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) in a subject comprising obtaining a tissue sample from a subject receiving a cancer treatment regimen and measuring the expression level of the long non-coding RNA for TROLL-8; wherein when the expression level of lncRNA for TROLL-8 is i) higher than a negative control and/or equivalent to or has not decreased relative to a positive control; indicates that the treatment regimen is not efficacious; and wherein the method further comprises changing the treatment regimen when the treatment regimen is not efficacious. In some aspects, the cancer comprises a cancer with a KRAS^{G12C} mutation or p53 mutation.

Also disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)) in a subject comprising i) obtaining a tissue sample from the subject; ii) assaying the tissue sample for the presence and/or expression level of the long non-coding RNA for TROLL-8; wherein the presence of lncRNA for TROLL-8 indicates the presence of a cancer in the tissue sample from the subject; and iii) administering to a subject an agent that knocks down expression of TROLL-8 or increases expression of carnitine palmitoyltransferase 1A (CPT1A).

In some aspects, the cancer comprises a cancer with a KRAS^{G12C} mutation or p53 mutation.

In one aspect, disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)), wherein expression of TROLL-8 is knocked down through the administration of one or more RNA-targeted therapeutics including, but not limited to antisense oligonucleotides, siRNA (such as, for example, SEQ ID NO: 2, SEQ ID NO: 3, and/or SEQ ID NO: 4), shRNA, ribozymes, transcription activator-like effector nucleases (TALEN), zinc finger nucleases (ZFNs) and/or clustered regularly interspaced short palindromic repeats/associated (CRISPR/Cas) nucleases.

Also disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)), wherein the treatment comprises administering to the subject carnitine palmitoyltransferase 1A (CPT1A) or a vector that overexpresses CPT1A.

In one aspect, disclosed herein are methods of treating, inhibiting, reducing, decreasing, amelioration, and/or preventing a cancer and/or metastasis (such as, for example, breast cancer, including, but not limited to triple negative breast cancer (TNBC) or invasive ductal carcinoma (IDC)), further comprising the administration of a second anti-cancer agent and/or immunotherapy. It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: TAp63 Regulated Oncogenic Long Non-Coding RNA-8 (TROLL-8) Regulates Human Breast Cancer Progression Through CPT1A Mediated Fatty Acid Oxidation

Metabolic reprogramming is a characteristic in cancer cells to rewire their metabolism for supporting a higher nutrient demand and defensing against oxidative stress to proliferate, invade and metastasize. Because the reprogramming is needed in each step of cancer progression, altered metabolism is now considered a core hallmark of cancer. Moreover, tumor cells reprogram mitochondria to meet the challenges of anabolic and catabolic requirements. Importantly, mitochondrial respiration and function are shown essential for tumor growth. Mechanistically, a variety of intrinsic and extrinsic factors influence metabolic reprogramming of cancer cells, including intracellular signaling pathways and their components, nutrient composition, oxygen availability, and acidity, respectively. Consequently, metabolic reprogramming renders cancer cells more vulnerability to metabolic targeting. Elucidating the mechanisms underlying cancer cell metabolism adaptation can help identifying cancer targets and developing new strategies.

Breast cancer is a heterogeneous disease, classified into several fundamentally different subtypes, namely luminal A, luminal B, human epidermal growth factor receptor 2 (HER2), and basal-like or triple-negative. Triple-negative breast cancer (TNBC) is an inherent aggressive tumor with triple-negative receptor status (estrogen receptor (ER), progesterone receptor (PR), and HER2) and thus, is not amenable to conventional targeted therapy. Patients diagnosed with TNBC carry relatively poor prognosis due to the lack of effective targeted therapy and resistance to chemotherapy, which is the only therapeutic of systemic treatment though. Cancer cells execute significant metabolic reprogramming to support cancer progression. Hence, new metabolic strategies are in urgent need for TNBC treatment. TNBC displays alterations in oncogenes, which direct the metabolic rewiring in multiple facets of cellular metabolism, including glycolysis, oxidative phosphorylation (OXPHOS), amino acid metabolism, and lipid metabolism in a reciprocal way. The metabolic reprogramming then results in a metabolic heterogeneity and plasticity of TNBC to better adapt and survive the surrounding microenvironment during progression. For example, TNBC displays elevated glycolytic enzymes, transporters, fatty acid oxidation (FAO), and glutaminolysis pathways to meet its bioenergetic and biosynthetic demands. Activated AMPK pathway induces mitochondrial enzymes involved in fatty acid oxidation (FAO) and glutaminolysis to facilitate the switch between glycolysis and OXPHOS. The combination of systemic metabolic targeting and chemotherapy can perform better anti-tumor effects and improve outcomes for patients with TNBC.

Long non-coding RNAs (lncRNAs) are a class of regulatory RNA transcripts longer than 200 nucleotides with no protein coding potential. Tens of thousands of lncRNAs have been identified across the non-protein coding regions of human genome, which accounts for more than 98% of all sequences, but vast majority of them remains to be functionally characterized in biological processes, especially cancer progression. Emerging molecular mechanisms of lncRNAs in regulating cancer metabolic reprogramming have been realized, LncRNA AGAP2-AS1 activates fatty acid oxidation through inducing CPT1 expression to promote stemness and trastuzumab resistance in HER2 positive breast cancer patients. LncRNA UCA1/miR-182 axis interacts with the fructose-2,6-bisphosphatase PFKFB2 to induce a glycolytic phenotype and mediate invasion of glioma cells. Overexpression of lncRNA UCA1 promotes mitochondrial function and ATP production in bladder cancer via miR-195 downregulation and ARL2 upregulation. Overexpression of lncRNA XLOC_006390 blocks c-Myc ubiquitination and stabilizes c-Myc to activate glutamate dehydrogenase 1 (GDH1) and promote glutamate metabolism. The molecular mechanisms that control how lncRNAs regulate cellular processes rely on their interactions with cellular macromolecules, including DNA, chromatin, RNA species, and proteins. Through these interactions, lncRNAs exert their regulatory functions via regulation of gene expression at multiple levels, including gene transcription, mRNA processing at the post-transcriptional level, protein translation and post-translational alterations such as phosphorylation, ubiquitination, and acetylation. LncRNA-protein interactions participate in the multiple regulatory levels. For example, at the transcriptional level, lncRNAs interact with histone methyltransferase, or histone demethylase, or acetylation enzymes, or DNA methyltransferase to regulate histone modifications and DNA methylation. LncRNAs can also directly bind to transcription factors to regulate gene expression or block the binding of negative transcriptional regulators to transcription factors and enhance gene expression. Post-translational modifications change protein expression, activity, and stability. LncRNAs mediate the binding of proteins to phosphatase or kinase to regulate protein phosphorylation; or serve as scaffold molecules to bring together ubiquitin ligases with their protein substrates to promote ubiquitination; or block the binding of negative regulator to deacetylase, regulating the activity of deacetylase and protein acetylation. Based on the understanding of lncRNA functions in metabolic reprogramming and the importance of lncRNA-protein interactions for their regulatory functions, it is critical to determine the interactions between cancer-associated metabolism reprogramming and the regulation of key metabolism-related proteins by lncRNA-protein interactions in TNBC. In the current study, we aimed to explore the role of lncRNA-protein interaction in mediating the post-translational modification, activity, and metabolism pathway of key metabolism-related enzyme in TNBC cells.

This study showed that TROLL-8, an oncogenic lncRNA interacts with a key enzyme in the AMPK signaling pathway, CPT1A, to regulate CPT1A activity and acetylation, through blocking the interaction between CPT1A and the acetyltransferase ACAT1, and affect fatty acid oxidation (FAO) in TNBC cell line CA1D. We further uncovered that CPT1A acetylation reduces its activity in long chain fatty acid transportation across mitochondrial membrane and consumption in the mitochondria. Moreover, TROLL-8 negatively correlates with TNBC patient overall survival rates and highly expresses in breast cancer compared to normal breast tissue, revealing an oncogenic role of TROLL-8 in TNBC. Collectively, the data suggested that TROLL-8-CPT1A interaction regulates the post-translational modification of CPT1A through blocking the interaction between CPT1A and ACAT1 and modulates CPT1A activity, resulting in altered energy metabolism in FAO in TNBC cell line. These findings implicate that TROLL-8 can serve as an indicator for TNBC diagnostics and the TROLL-8/CPT1A/ACAT1 axis might be targeted for TNBC therapy.

### a) Results

(1) TAp63 Regulated Oncogenic lncRNA-8 (TROLL-8) Expression Positively Correlates with Human Breast Cancer Progression

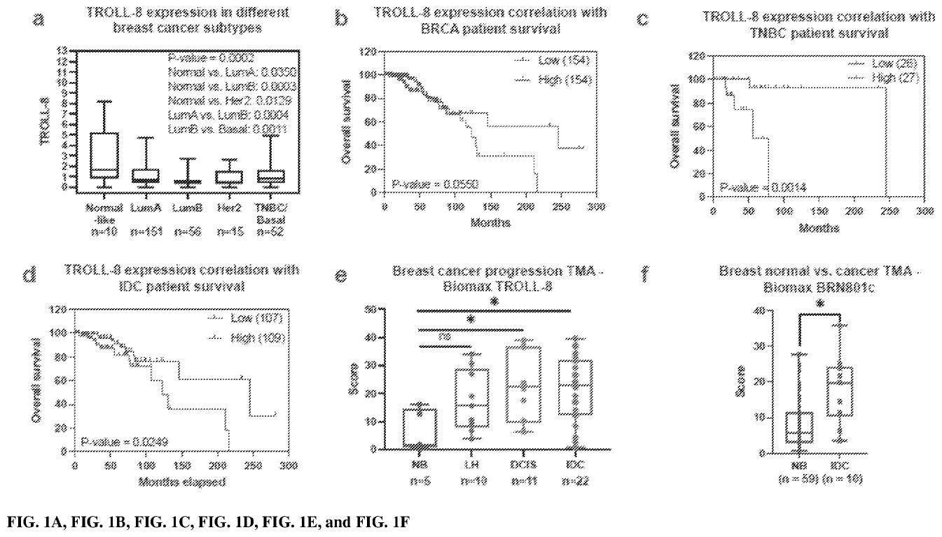

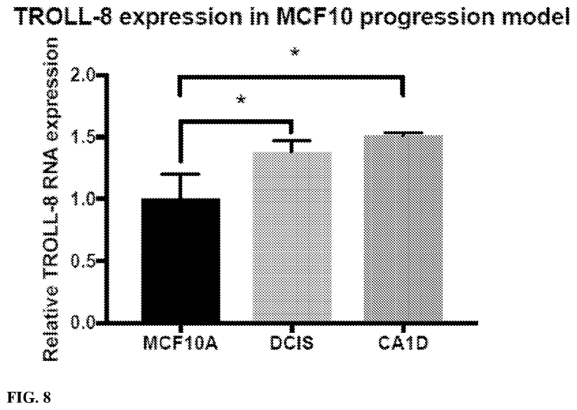

We have identified 9 TAp63-regulated oncogenic lncRNAs, named TROLLs (TAp63 regulated oncogenic lncRNAs). In this paper, we focused on lncRNA TROLL-8, named for TAp63 regulated oncogenic lncRNA 8. Importantly, TROLL-8 knockdown by siRNA significantly promoted breast cancer cell apoptosis, indicating a role in breast cancer progression. Pan-cancer analysis of TROLL-8 expression in TCGA database indicated that TROLL-8 expression is significantly higher in basal/TNBC breast cancer subtype (FIG. 1a). Moreover, TROLL-8 expression negatively correlates with breast cancer patient overall survival rate, and the correlation almost reaches significance (FIG. 1b). Specifically, TROLL-8 expression is negatively correlated with breast cancer subtype patient overall survival rate, including TNBC and invasive ductal carcinoma (IDC) patients (FIGS. 1c and 1d). These data indicate an oncogenic role of TROLL-8 in TNBC and IDC molecular subtypes. To check if the oncogenic role of TROLL-8 from bioinformatics analysis with TCGA datasets can be recapitulated in clinical cases, we performed human breast tissue microarray—TMA with ISH assay. TROLL-8 expression is significantly higher in breast cancer patient samples, including ductal carcinoma in situ (DCIS) and IDC when compared to normal breast (NB) tissues (FIGS. 1e and 1f). In the TNBC cell lines—MCF10 progression model, TROLL-8 expression is higher in the tumorigenic DCIS cell and metastatic CA1D cell when compared to the normal epithelial MCF10A cell (FIG. 8). These data indicate an oncogenic role of TROLL-8 in human breast cancer progression.

(2) TROLL-8 Interacts with Proteins Enriched in Cellular Metabolism

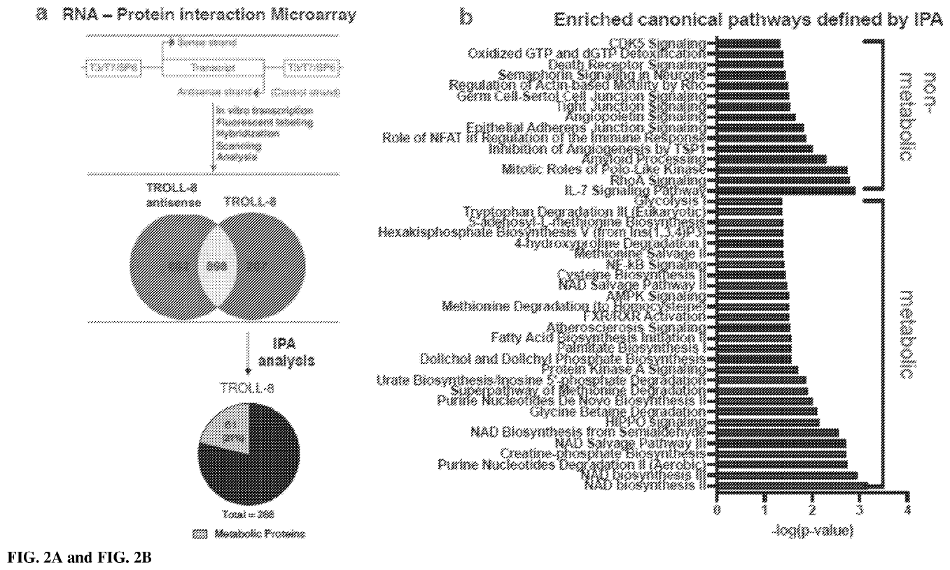

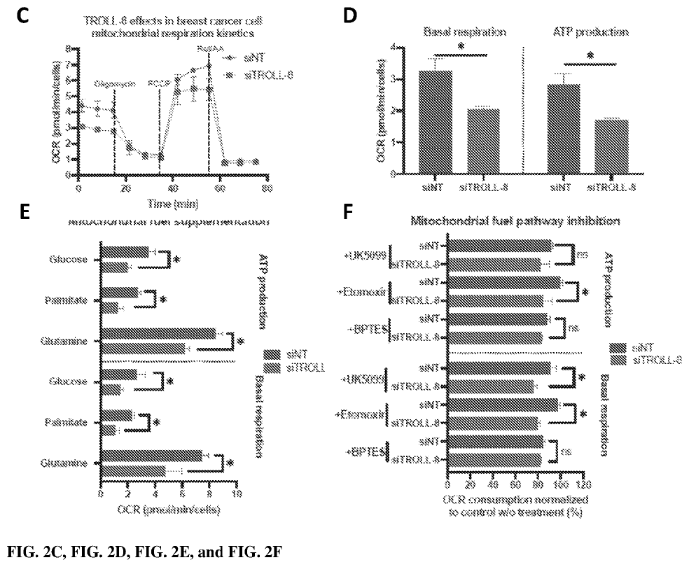

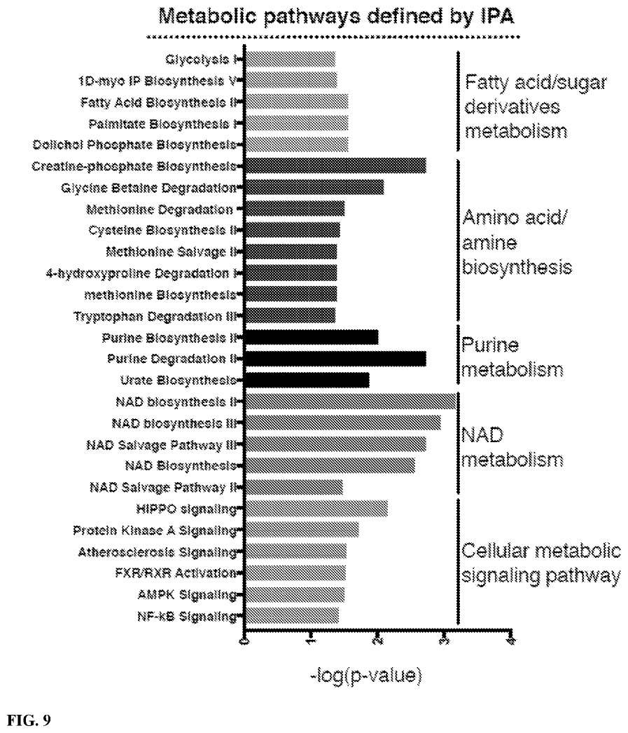

LncRNAs function mainly through interactions with proteins, either signaling proteins or regulatory proteins. The commercially available protein microarray—Protoarray Human Protein Microarray Version 5.0 (Thermo Fisher Scientific) provides over 9,400 unique, full-length human recombinant proteins spotted in duplicate on a nitrocellulose covered glass slide, to screen for novel protein biomarkers in diseases or map protein interactions with other macromolecules important to biological pathways. To figure out the potential of TROLL-8 in breast cancer progression, we performed the protein microarray experiments to identify TROLL-8 interacting proteins and Ingenuity Pathway Analysis (IPA) to reveal the canonical signaling pathways, in which those interacting proteins are involved. As in the workflow described in FIG. 2a, we in vitro transcribed, labeled and hybridized the TROLL-8 RNA strand and its antisense strand (the internal control) to the Protoarray slides to screen for the candidate proteins that specifically interact with TROLL-8. As a result, we identified 288 specific interacting proteins for TROLL-8 RNA. 21% of these proteins (61 proteins) are metabolic proteins. IPA pathway analysis demonstrated that these 288 proteins are enriched in metabolic pathways (FIG. 2b). These 61 metabolic proteins can be categorized to 5 groups: fatty acid/sugar derivatives metabolism, amino acid/amine biosynthesis, purine metabolism. NAD metabolism and cellular metabolic signaling pathway (FIG. 9).

To define the potential of TROLL-8 in regulating human breast cancer cell metabolism, seahorse metabolic assays were performed to test the effect of TROLL-8 in breast cancer mitochondrial respiration. We targeted TROLL-8 with siRNA in the CA1D cells. With the seahorse mitochondrial stress test assay, we found that TROLL-8 silencing significantly reduced the mitochondrial respiration (FIG. 2c), specifically, the basal respiration and ATP production (FIG. 2d). There are three types of mitochondrial fuels oxidized and used by live cells for energy production. Modulation of these fuel sources can affect glucose, amino acid, or lipid homeostasis which becomes dysfunctional in diseases like cancer.

To pinpoint the specific fuel oxidation pathways that are regulated by TROLL-8 during metabolic stress, we supplemented cells with individual fuels, either glucose, glutamine, or palmitate and found that TROLL-8 silencing leads to significantly reduced basal respiration and ATP production (FIG. 2e), indicating that with nutrient limitation, TROLL-8 affects all three fuel metabolism pathways in the mitochondria. Complementarily, inhibition of enzymes and transporters driving these mitochondrial oxidation pathways is critical to understand how substrate utilization and metabolic activity are reprogrammed. To determine and compare the mitochondrial capacity and dependency for fatty acid, glutamine, and glucose oxidation after TROLL-8 silencing under physiological condition, we employed individual small molecule inhibitors in the mitochondrial stress test assay: UK5099, which inhibits the mitochondrial pyruvate carrier (MPC) and targets the glucose oxidation pathway; Etomoxir (Eto), which inhibits the fatty acid transporter CPT1A and targets the fatty acid oxidation (FAO) pathway; BPTES, which inhibits glutaminase (GLS1) and targets the glutamine oxidation pathway. We treated CA1D cells with or without inhibitors and applied inhibitors to either non-treated (siNT) or TROLL-8 silencing (siTROLL-8) CA1D cells. OCR was inhibited and normalized to their corresponding non-treated groups that have no inhibitor application. We noticed that TROLL-8 depletion increased the impact of FAO and glucose oxidation inhibitors in basal respiration; but not the glutamine oxidation inhibitor, indicating that cells lacking TROLL-8 rely on FAO and glucose oxidation for mitochondrial respiration. Meanwhile, we also found that cells lacking TROLL-8 also rely on FAO for ATP production. Data combining the fuel supplementation assay and fuel oxidation pathway inhibition assay implicate that TROLL-8 regulates glucose, fatty acid, and glutamine oxidation for energy metabolism.

### (3) TROLL-8 Downregulation Leads to Compromised Fatty Add Oxidation (FAO) and Long-Chain Fatty Adds (LCFAs) Accumulation

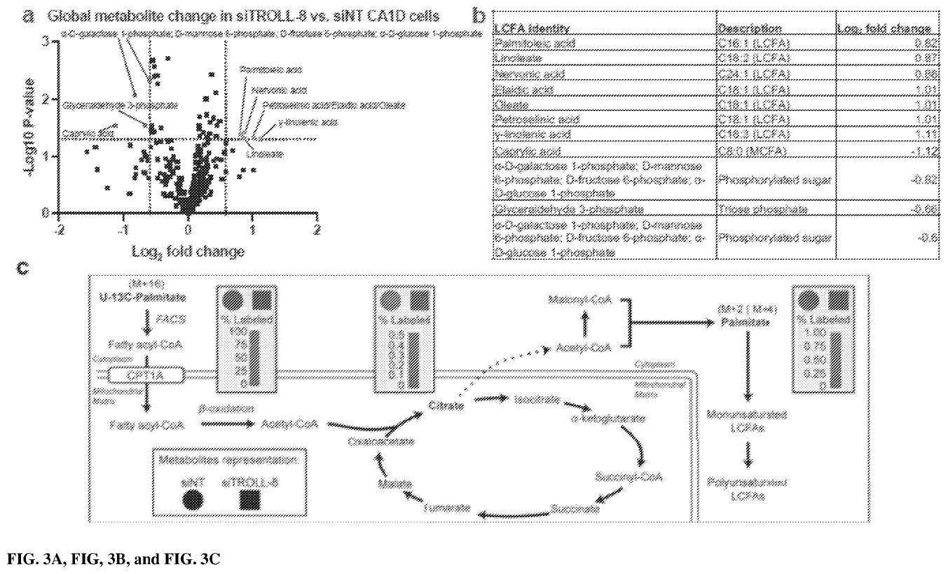

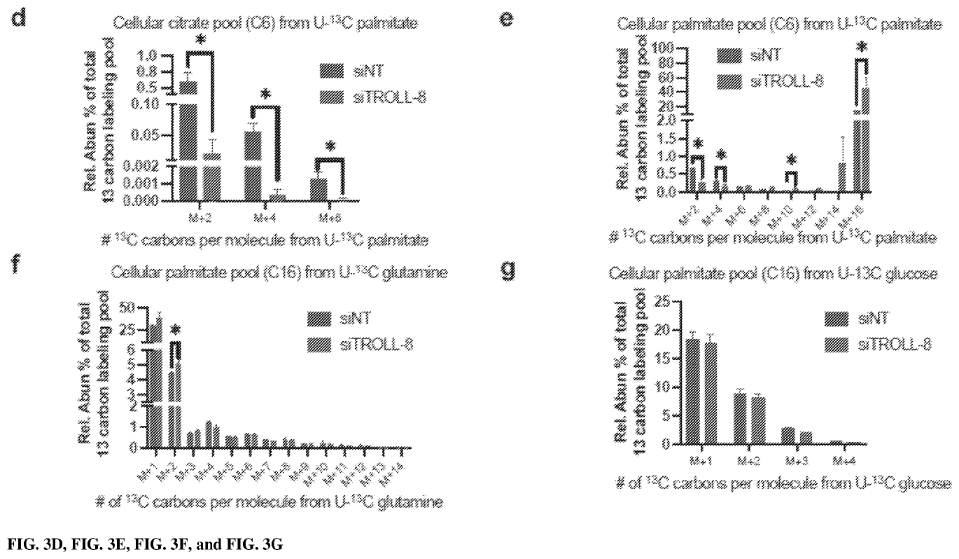

To characterize how TROLL-8 regulates enzymes, intermediates, and metabolites in mitochondrial fuel oxidation pathways, we performed a global liquid chromatography-mass spectrometry (LC-MS) targeted in the tricarboxylic acid (TCA) cycle, glucose, fatty acid, and glutamine oxidation pathways. Multiple metabolites significantly affected by TROLL-8 silencing with over 1.5-fold change in concentration were found (FIG. 3a red and green dots). Very interestingly, all the metabolites that are significantly upregulated in siTROLL-8 CA1D cells are LCFAs (FIG. 3b). The downregulated metabolites include medium chain saturated fatty acid, e.g., caprylic acid, and sugar phosphates, e.g., glyceraldehyde 3-phosphate and alpha-D-galactose 1-phosphate.

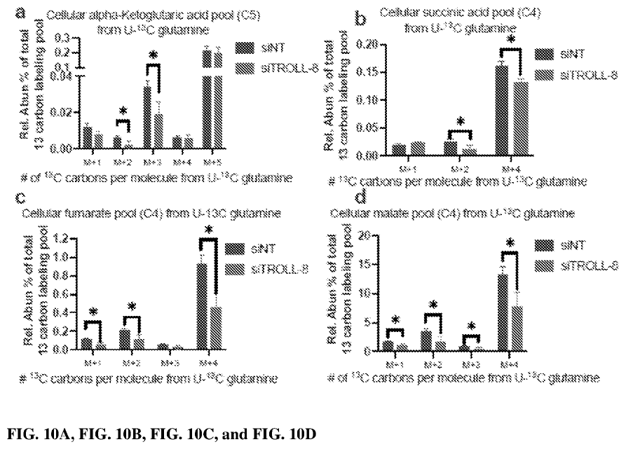

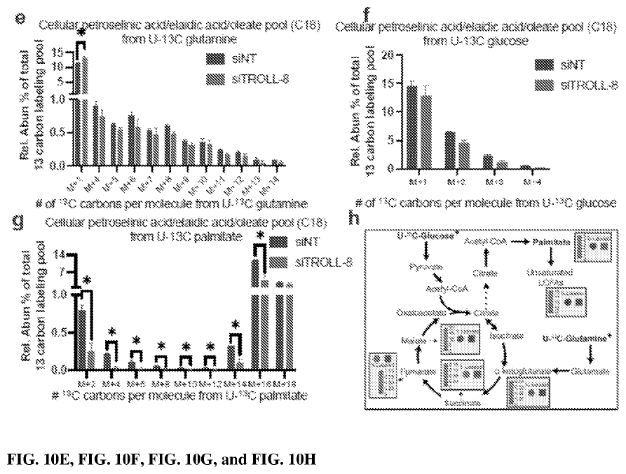

Fatty acids serve as important elements in cellular membrane structure, energy storage and signaling pathway components. Dysregulated fatty acid metabolism has been associated with various prevalent diseases, including cancer. The cellular fatty acid pools are formed by a combination of series events, including de novo fatty acid synthesis from acetyl-CoA as the substrate, elongation using acetyl-CoA as the substrate, and desaturation reactions. The mitochondrial fuels contribute to the cellular acetyl-CoA precursor generation. Glucose-derived pyruvate enters the mitochondria and is converted to acetyl-CoA. Glutamine is metabolized to acetyl-CoA through glutaminolysis or reductive carboxylation (FIG. 10H). Cells take up fatty acids and convert them to fatty acyl CoA, which is transported by CPT proteins into mitochondria for oxidation and acetyl-CoA production (FIG. 3c). ¹³C-labeled precursors that are metabolized to acetyl-CoA in combination with mass spectrometry (LC-MS) is a powerful tool for providing information on the contribution of individual precursors in fatty acid metabolic reactions. To further understand which mitochondrial fuel oxidation pathway specifically contribute to the accumulated LCFAs after TROLL-8 depletion, we performed heavy carbon tracing experiments with uniformly labeled glucose (U-¹³C-glucose), glutamine (U-¹³C-glutamine), or palmitate (U-¹³C-palmitate). U-¹³C labeled precursors are metabolized to U-¹³C-acetyl-CoA. De novo synthesized LCFAs are built by incorporation of U-¹³C-acetyl-CoA. FAO is directly assessed by feeding CA1D cells with U-¹³C-palmitate and measuring the labeling of FAO products. The product of FAO is acetyl-CoA. U-¹³C-palmitate is degraded to ¹³C-acetyl-CoA (the M+2 isotopologue), which then reacts with oxaloacetate in the TCA cycle to produce ¹³C-citrate. The level of ¹³C-citrate (the M+2|M+4|M+6 isotopologue) indicates the efficiency of FAO. Upon TROLL-8 silencing, ¹³C-citrate labeling from U-¹³C-palmitate decreased by over 90%, indicating that TROLL-8 silencing effectively blocks FAO (FIG. 3d). Moreover, we noticed significantly increased levels of fully labeled ¹³C₁₆-palmitate (U-¹³C-palmitate) (FIG. 3e), which is consistent with the results of the global LC-MS experiments shown in FIG. 3a. However, we noticed much less amount of isotopologues with fewer labeling, e.g., M+2, . . . , M+14, when compared to the fully labeled ¹³C₁₆-palmitate. Moreover, siTROLL-8 group displays significantly less amount of the de novo synthesized M+2 and M+4 isotopologues of ¹³C-palmitate and petroselinic acid/elaidic acid/oleate formed by elongation and desaturation from U-¹³C-palmitate (FIG. 10G). These data implies that compromised FAO, not the de novo synthesis, contributes to the LCFA accumulation induced by TROLL-8 depletion.

The glutamine metabolism is another important contributor for both cellular acetyl-CoA and the following de novo fatty acid synthesis. In glutamine metabolism, the imported U-¹³C-glutamine undergoes two main pathways for energy production: glutaminolysis and reductive carboxylation. The glutaminolysis pathway intermediates showed an overall reduction in the labeled isotopologues, including alpha-ketoglutaric acid, succinic acid, fumarate, and malate (FIG. 10A-10D), indicating a reduction in glutaminolysis route for glutamine metabolism. However, TROLL-8 silencing did induce an increase in the level of ¹³C-palmitate labeled from U-¹³C-glutamine (FIG. 3f and FIG. 10E), indicating that TROLL-8 knockdown leads to the compromised glutaminolysis and LCFA accumulation. U-¹³C-glucose tracing detected several LCFAs incorporated with U-¹³C₂-acetyl-CoA. However, we did not notice a significant change in the level of LCFAs metabolized from U-¹³C-glucose after TROLL-8 depletion (FIG. 3g and FIG. 10F), implying that glucose oxidation did not contribute to the LCFA accumulation detected by the global LC-MS after TROLL-8 depletion. To summarize, isotope tracing with carbon-13 showed that TROLL-8 silencing compromised FAO and glutaminolysis, and the U-¹³C-palmitate was dramatically accumulated in siTROLL-8 vs. siNT (40% vs. 10%). These data indicates that the accumulated LCFAs is due to reduced FAO.

(4) TROLL-8 Interacts with the FAO Rate-Limiting Enzyme, CPT1A

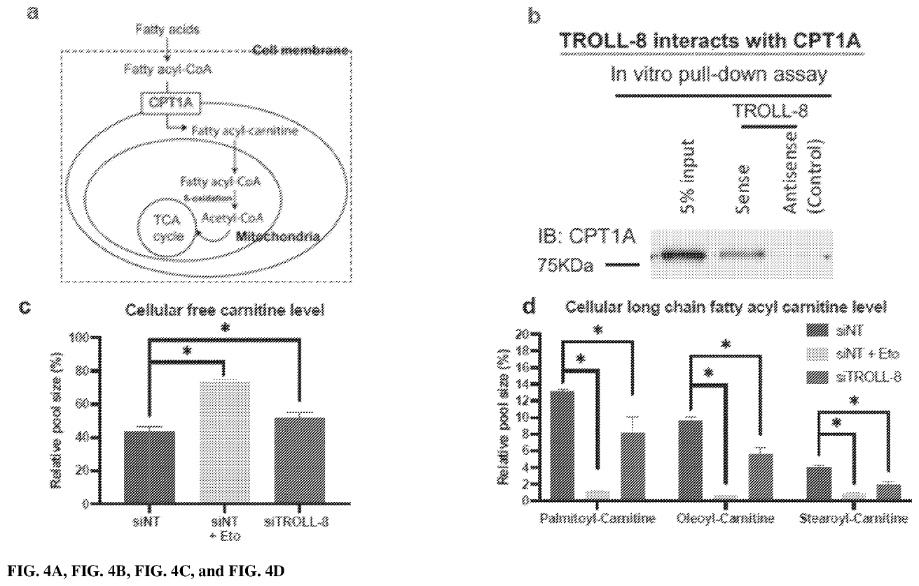

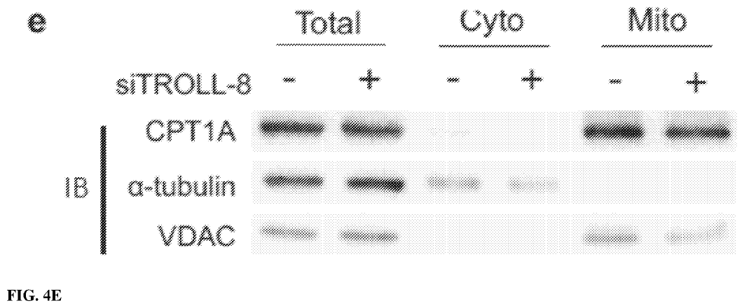

Mechanistically, to figure out how TROLL-8 silencing compromised FAO, we referred to the 61 metabolic protein candidates identified by the protein microarray experiment, to select metabolic proteins that play critical roles in FAO. We selected protein(s) based on the following criteria: the protein(s) are involved in fatty acid metabolism; the protein(s) are enzymes; there are activity assays for the protein(s) available; the protein(s) are ‘druggable’. Following the above criteria, we identified CPT1A, carnitine palmitoyltransferase 1A, for further functional studies. CPT1A connects carnitine to LCFAs and converts them to long-chain fatty acyl-carnitine, which can then cross the inner membrane of mitochondria for oxidation and energy production (FIG. 4a). Thus, CPT1A is a critical and rate-limiting enzyme in the process of fatty acid oxidation. To confirm the direct physical interaction between CPT1A protein and TROLL-8 RNA, we performed a biotinylated RNA-pull down assay with in vitro transcribed and biotinylated TROLL-8 and CPT1A in CA1D cells. TROLL-8, not its antisense RNA strand, which serves as the internal structure control, pulled down CPT1A proteins in CA1D cells (FIG. 4b), indicating a specific interaction between TROLL-8 RNA and CPT1A protein.

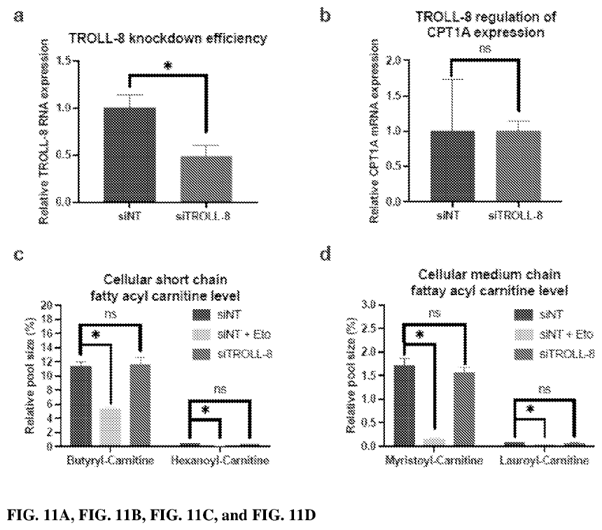

CPT1A belongs to the carnitine palmitoyl transferase family and its deficiency leads to a rare disease with autosomal recessive metabolic disorder of long-chain fatty acid oxidation (FAO). CPT1A is highly expressed in cancer cells such as breast, prostate, ovarian, and lung cancers. To study how TROLL-8/CPT1A interaction affects CPT1A-mediated FAO and characterize the underlying mechanism of CPT1A as a downstream effector of TROLL-8 function, we performed a comprehensive characterization of the regulation of CPT1A by TROLL-8 at the transcriptional level, translational level, post-translational level, and enzymatic activity. Utilizing the metastatic human breast cancer cell line CA1D, we targeted TROLL-8 with siRNA. CPT1A converts fatty acyl-CoA to fatty acylcarnitine in the presence of cytosolic carnitine in the process of transporting fatty acid across the outer mitochondrial membrane. To test CPT1A activity regulation by TROLL-8, we performed a LC-MS experiment with the following groups of CA1D cells: siNT, siTROLL-8, and siNT+Eto. We saw that both Eto and TROLL-8 silencing increased the level of carnitine, which is the substrate for CPT1A activity (FIG. 4c). Treatment with CPT1A inhibitor Eto significantly reduced the levels of short-chain (FIG. 11C), medium-chain (FIG. 11D), and long-chain fatty acylcarnitine (FIG. 4d), indicating a reduced CPT1A activity on all types of fatty acylcarnitine species. Eto is an irreversible CPT1-specific inhibitor, which binds to the active site with a covalent bond to block CPT1A-fatty acyl-CoA complex formation. Unlike Eto, TROLL-8 silencing significantly reduced long-chain fatty acylcarnitine levels (FIG. 4d), but not short-chain (FIG. 11C) or medium-chain species (FIG. 11D), demonstrating that TROLL-8 regulates CPT1A enzyme activity, focusing on long-chain fatty acid transportation. Unlike the irreversible inhibition induced by Eto treatment, TROLL-8 interaction can induce intermediate events, leading to an allosteric modulation of CPT1A activity with reduced affinity for long-chain fatty acyl-CoA at the active site of CPT1A. However, compared to the non-targeted group, depletion of TROLL-8 did not affect CPT1A mRNA level (FIGS. 11A and 11B) or protein level (FIG. 4g), either in the mitochondria, or cytosol, or the whole cellular compartment. Moreover, there is no cytosolic translocation of CPT1A, indicating that TROLL-8 silencing did not change CPT1A protein expression. TROLL-8 can mediate CPT1A function at the post-translational level.

### (5) TROLL-8 Regulated CPT1A Post-Translational Modifications Mediate its Activity

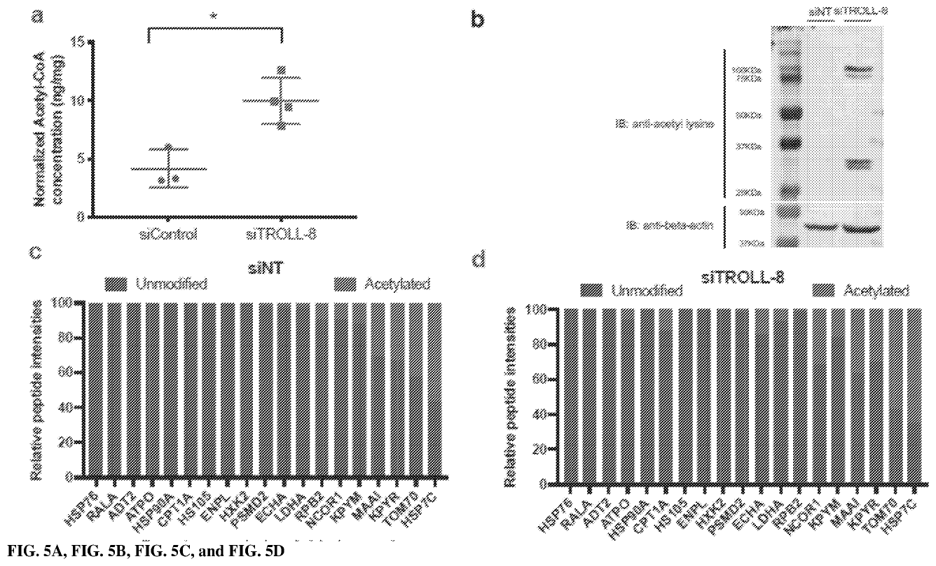

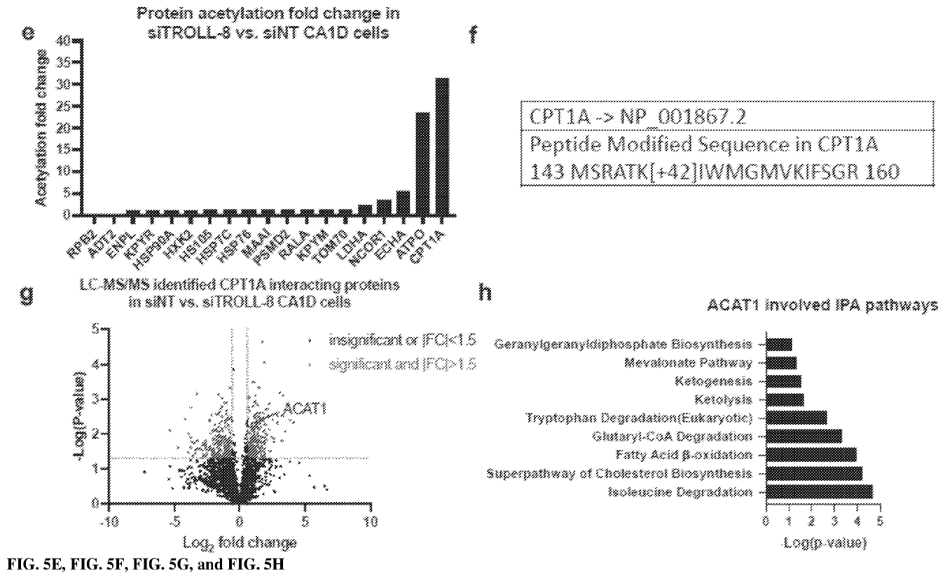

Acetylation, a reversible covalent modification, has been shown to regulate metabolic protein activity through either causing conformation changes in the active site, or blocking substrate binding to the enzyme. Previous acetylation proteomics studies identified over 2,000 acetylated proteins in mammalian cells and among them, a large portion of mitochondrial proteins are reversibly acetylated at the lysine site. Over 50% of the proteins in fatty acid metabolism, sugar metabolism and amino acid metabolism are acetylated, with fatty acid metabolism as the top acetylation enriched pathway. Thus, TROLL-8 interaction can cause CPT1A acetylation, leading to an allosteric regulation of CPT1A activity. Moreover, we noticed that TROLL-8 depletion significantly reduced the oxidation of all three mitochondrial fuels, which all produce acetyl-CoA in the mitochondria. As we know, acetyl-CoA is the precursor for protein acetylation. To test if TROLL-8 regulates CPT1A activity post-translationally, first, we performed LC-MS to test and compare the total cellular acetyl-CoA level in non-treated and TROLL-8 depleted CA1D cells (FIG. 5a). We noticed that TROLL-8 silencing significantly increased cellular acetyl-CoA level, which can induce global protein hyperacetylation. We ran western blot with pan anti-lysine acetylation antibody to detect how TROLL-8 silencing affects protein acetylation with cell lysates from non-treated and siTROLL-8 treated CA1D cells (FIG. 5b). We noticed that TROLL-8 depletion caused global protein hyperacetylation, indicating a role of TROLL-8 in protein post-translational modification.

Next, we executed LC-MS with the same samples as the western blot to perform proteomic analysis of the acetylated peptides with lysine acetylation sites in non-treated (FIG. 5c) and siTROLL-8 treated (FIG. 5d) groups. 20 metabolic proteins with varied lysine acetylation were demonstrated. Acetylation fold change was calculated by dividing the relative acetylated peptide intensities in TROLL-8 depleted cells by that in non-treated cells (FIG. 5e) and we found that TROLL-8 deletion changed global protein acetylation levels, especially CPT1A, the top one hyper-acetylated protein, with 35-fold more acetylation after TROLL-8 silencing. The acetylation site of CPT1A after TROLL-8 depletion is K148 (FIG. 5f). The acetylation status of a given protein is determined by the balance in the action of acetyltransferase and deacetylase to add or remove the acetyl groups from the lysine residues, respectively.

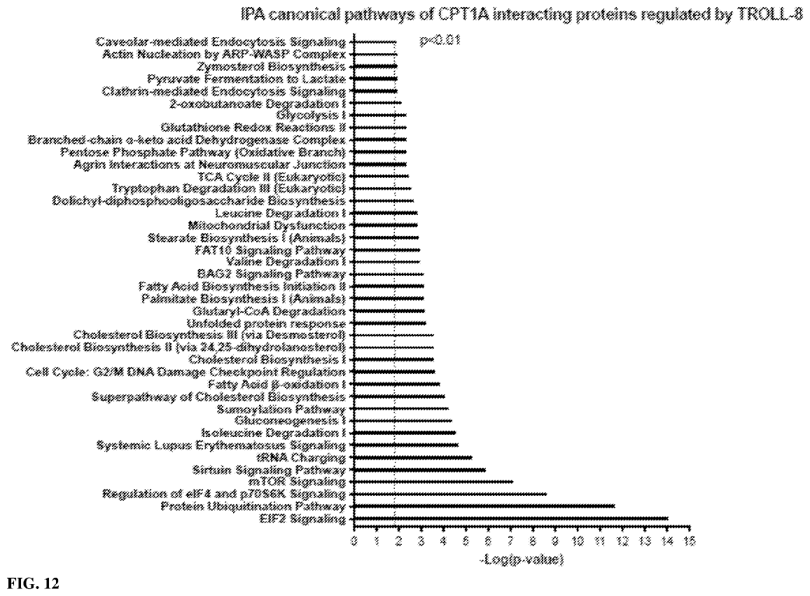

To identify enzymes that are responsible for CPT1A hyperacetylation, we performed a LC-MS to characterize acetyltransferase(s) that show higher binding to CPT1A or deacetylase(s) which lose affinity for CPT1A in TROLL-8 depleted CA1D cells (FIG. 5g). Interestingly, we identified that ACAT1, the acetyl-CoA acetyltransferase 1 protein, are co-immunoprecipitated with CPT1A by around 2-fold more in TROLL-8 deleted CA1D cells when compared to the non-treated cells. IPA pathway analysis demonstrated that ACAT1 participates in cellular metabolism, including fatty acid metabolism (FIG. 5h). Very interestingly, IPA pathway analysis showed that the canonical pathways of the proteins with significant changed affinity for CPT1A (|FC|>1.5) after TROLL-8 silencing are enrich in cellular metabolism (FIG. 12), indicating that TROLL-8 silencing induces significant affinity change between CPT1A and metabolic proteins.

### (6) Expression of CPT1A and its Hypo-Acetylated Form Restore TROLL-8 Knockdown to Induce Mitochondrial Respiration and Tumorigenesis in Breast Cancer Cells

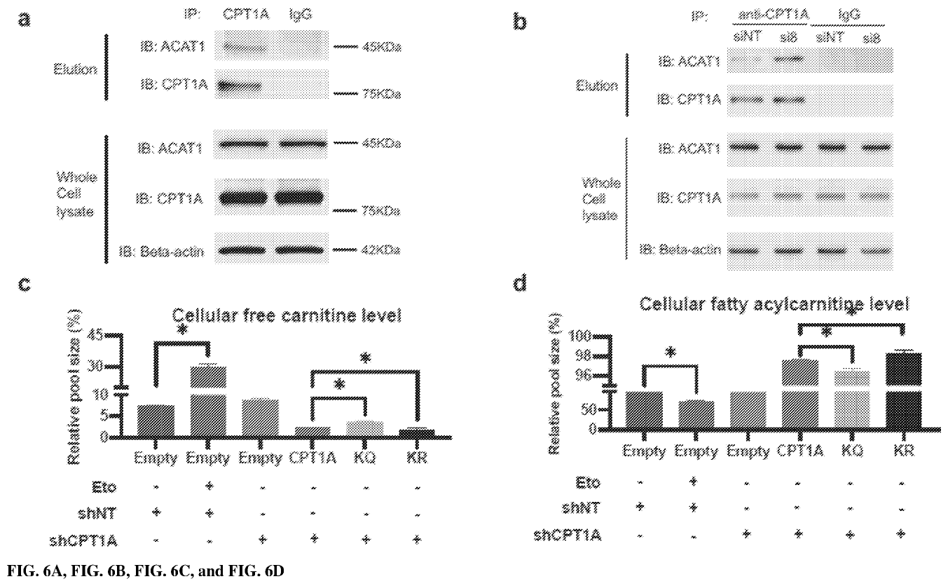

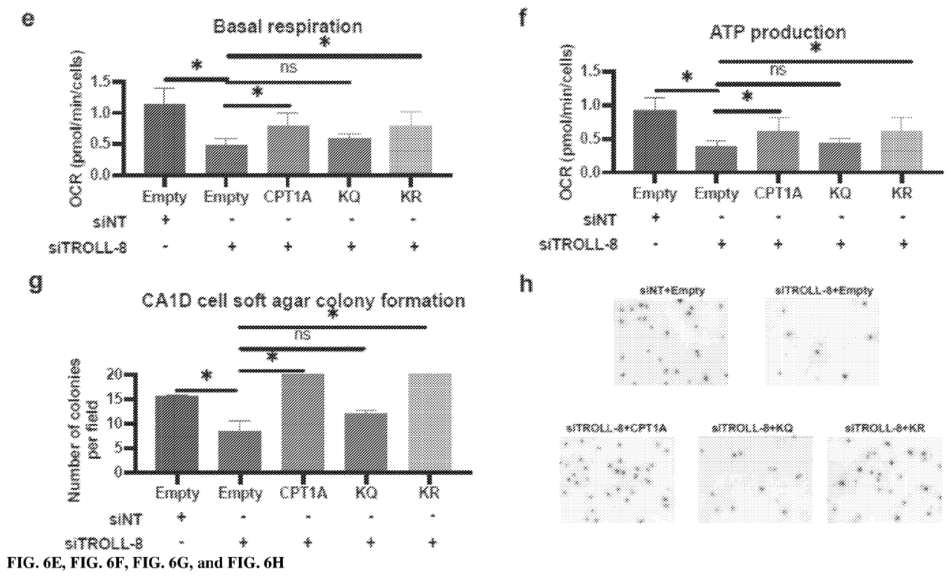

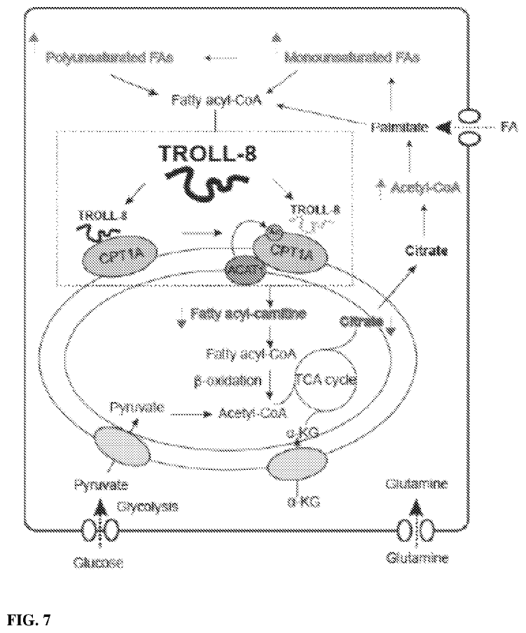

ACAT1, acetyl-CoA acetyltransferase, has been shown to have thiolase activity in isoleucine degradation, ketogenesis and fatty acid oxidation. Beyond its classical activity, ACAT1 acetylates the Pyruvate Dehydrogenase Phosphatase Catalytic Subunit1 (PDP1) and the Pyruvate Dehydrogenase E1 Subunit Alpha1 (PDHA1) in the Pyruvate Dehydrogenase Complex (PDC), which negatively regulates the activity of Pyruvate Dehydrogenase (PDH) in glycolysis and tumor growth. To confirm that ACAT1 shows higher binding to CPT1A after TROLL-8 depletion, we performed immunoprecipitation (IP) and western blot (WB) to check if ACAT1 can specifically co-immunoprecipitate (co-IP) with CPT1A in CA1D cells (FIG. 6a) and if TROLL-8 silencing increase their interaction (FIG. 6b). In the experiment, we have the following groups: whole cell lysate (5% of the total input); cell lysate from either CA1D cells, or siNT/siTROLL-8 treated CA1D cells immunoprecipitated with CPT1A specific antibody; or the same cell lysate immunoprecipitated with normal IgG control antibody. From the results of immunoblots probed with either CPT1A specific or ACAT1 specific antibodies, we saw that CPT1A specifically immunoprecipitated ACAT1 in the CA1D cells, indicating that CPT1A physically interacts with ACAT1. While there is no change in the expression level of either CPT1A or ACAT1 in the whole cell lysate, CPT1A specifically immunoprecipitated more ACAT1 (by around 2-fold) in siTROLL-8 CA1D cells compared to non-treated cells, confirming the LC-MS discovery that TROLL-8 depletion increased the physical interaction between CPT1A and ACAT1.

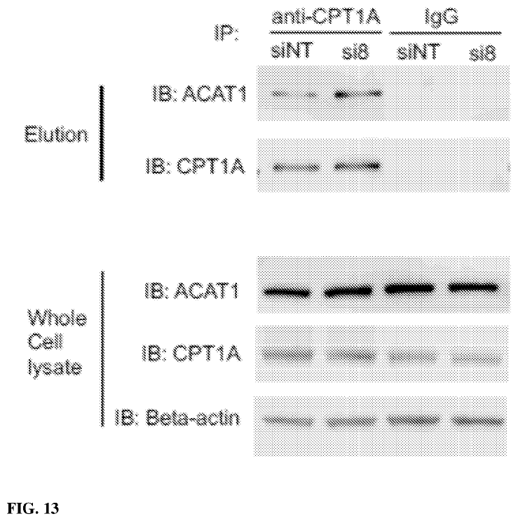

To define if this TROLL-8 regulated physical interaction also occurs in other TNBC cell line, we performed IP and WB with DCIS cells, either with siNT or siTROLL-8 treated (FIG. 13) and noticed that ACAT1 and CPT1A showed higher binding in siTROLL-8 treated DCIS cells, confirming TROLL-8 regulation of CPT1A/ACAT1 interaction in TNBC cells. Metabolic enzyme activity can be controlled by protein amount and catalytic activity. Acetylation has been shown to be involved in both aspects. Our data demonstrates that TROLL-8 silencing increased CPT1A acetylation, which can lead to the reduced CPT1A activity.

To characterize the inter-connection between CPT1A activity and its acetylation state, we hypothesized that CPT1A hyperacetylation leads to activity inhibition. To test this idea, we deleted the endogenous CPT1A by a shRNA targeting the 3′UTR of CPT1A mRNA and simultaneously overexpress either wild type (WT) or a mimic of acetyl lysine mutant (K to Q) or a mimic of non-acetylated lysine mutant (K to R) at the K148 site, which is detected by our LC-MS/MS proteomics analysis. With cell lysates from these groups: WT, KQ and KR, we performed LC-MS to detect carnitine and acylcarnitine levels to reflect CPT1A activity and determine how acetylation status of CPT1A affects its activity (FIGS. 6c and 6d). From the results, we saw that Eto treatment augmented the substrate, free carnitine level and reduced the product, fatty acylcarnitine level, indicating the reduced CPT1A activity. Overexpression of the hyperacetylated mutant (shCPT1A+KQ group) demonstrated significantly higher free carnitine level, and significantly lower fatty acylcarnitine level when compared to the CPT1A overexpression, indicating a decrease in CPT1A activity. Whereas compared to the CPT1A form, overexpression of the hypoacetylated mutant (shCPT1A+KR group) showed reduced free carnitine level and increased fatty acylcarnitine level, indicating an increase in CPT1A activity. These data implies that CPT1A acetylation regulates its activity and hyperacetylation induces a lower enzymatic activity.

We next investigated whether TROLL-8 functions through regulating CPT1A activity/acetylation. First, we conducted the seahorse mitochondrial stress test. TROLL-8 silencing significantly reduced mitochondrial basal respiration, ATP production in CA1D cells (FIGS. 6e and 6f). Overexpression of both the WT and KR forms of CPT1A significantly rescued the impaired mitochondrial basal respiration and ATP production. However, overexpression of the KQ hyperacetylated form of CPT1A did not rescue the impaired mitochondrial respiration. These data indicate that TROLL-8 regulates mitochondrial metabolism through CPT1A and CPT1A acetylation status affects its rescuing effects in TROLL-8 depletion impaired mitochondrial respiration. To identify the functions of TROLL-8/CPT1A axis in tumorigenesis, we performed the anchorage-independent soft agar assay and measured soft agar colony growth (FIGS. 6g and 6h). Compared to the siNT control group, TROLL-8 knockdown significantly reduced colony formation. In addition, overexpressing WT and KR CPT1A form in TROLL-8-knocked down cells significantly increased CA1D cell colony formation. However, overexpressing KQ CPT1A form did not rescue the impaired CA1D cell colony formation in TROLL-S-knocked down cells. Together, these data demonstrated that TROLL-8 functions as an oncogene via regulating CPT1A and CPT1A acetylation status regulates its contribution in TROLL-8 mediated cellular metabolism and tumorigenesis.

### b) Discussion

In spite of that a large number of lncRNAs have been identified and characterized to involve in breast cancer progression and cellular metabolism, the role of lncRNAs in bridging cellular metabolism and breast cancer progression remains elusive and it is important to explore the nature of this connection for developing effective therapeutic strategies in breast cancer progression. TAp63 is an isoform of the p53 family transcription factors, p63 and has tumor suppressive activities, which can be inhibited by mutant p53 binding. The data showed that TAp63 regulates two of cancer hallmarks. First, inhibiting cancer metastasis. Specifically, TAp63^{-/-} mice developed mammary adenocarcinoma, which spontaneously metastasized to the liver, lung, and brain. Mechanistic characterization shows that TAp63 coordinately regulates Dicer and miRNA to suppress metastasis. Second, deregulating cellular energetics. TAp63^{-/-} mice become obese by 8 months of age with high fat diet. They had increased body fat present underneath the skin and intercalated into multiple organs. Systematic assessment demonstrated that TAp63 transcriptionally activates proteins in the AMPK signaling pathway to regulate glucose and lipid metabolism. We identified 9 TAp63-regulated oncogenic lncRNAs, namely TROLLs (TAp63 regulated oncogenic lncRNAs), in human breast cancer progression. One of the TROLLs is MALAT1, previously defined to promote breast cancer metastasis where high level of lncRNA MALAT correlates with poor overall survival. Another two of the TROLLs activate the AKT pathway through regulating the subcellular translocation of AKT pathway component, WDR26, to promote breast cancer progression. With TAp63-regulated oncogenic lncRNAs in breast cancer progression being characterized, crosstalk between TROLLs and cellular metabolism in breast cancer progression needs to be elucidated. In this study, we demonstrate that the TAp63-regulated oncogenic lncRNA TROLL-8, regulates FAO through mediating the FAO rate-limiting enzyme CPT1A activity, acetylation and interaction with the acetyltransferase ACAT1. Expression of CPT1A (WT or KR forms) contributes to restore TROLL-8 knockdown impaired mitochondrial respiration and tumorigenesis in breast cancer cells, indicating that TROLL-8 regulates the crosstalk between FAO and tumorigenesis in breast cancer by regulating CPT1A activity and acetylation.

TROLL-8 is found to be an oncogenic lncRNA with reduced human breast cancer cell migration, invasion, and increased apoptosis when depleted. Using the analysis of clinical cases and TCGA datasets, we found that the expression of TROLL-8 is prognostic in breast cancer, especially in basal-like/TNBC and negatively correlates with the overall survival rate of TNBC and IDC breast cancer patients.

LncRNAs can act through binding to specific proteins. Understanding of the lncRNA interacting proteins and their downstream signaling pathways can provide a clue regarding lncRNA functions. For example, p53 mediates glucose metabolism in cancer progression and the loss of p53 leads to the promotion of glycolysis and mitochondrial respiratory damage and the suppression of TCA cycle.

lncRNA CUDR interacts with p53 mutant (N340Q/L344R) to form a complex, bind to the promoter of Pyruvate Kinase M2 (PKM2) and enhance its gene expression, leading to increased glycolysis in metabolic reprogramming. To identify TROLL-8-binding proteins, we developed human protein microarray analysis. The TROLL-8 sense, and antisense RNA were transcribed and labeled with Cy5 and independently hybridized to a protein microarray slide containing over 9,400 recombinant human proteins. IPA pathway analysis demonstrates that 21% of the specific interacting proteins and 67% of the enriched canonical pathways of these proteins are involved in cellular metabolism, indicating that TROLL-8 plays important roles in cellular metabolism. Specifically, TROLL-8 regulates mitochondrial respiration conducted by glucose, fatty acid (palmitate) and glutamine and TROLL-8 silencing induces LCFA accumulation. Isotopic tracing of mitochondrial fuel metabolism showed that TROLL-8 mediates long-chain fatty acid oxidation and glutaminolysis, a pathway for glutamine metabolism, which contributes to cellular acetyl-CoA; the LCFA accumulation induced by TROLL-8 silencing is due to compromised FAO.

CPT1A protein displayed the specific TROLL-8 binding and plays important roles to activate FAO in the mitochondria that increases ATP and NADPH, protecting cancer against the environment stress. Oxidation of exogenous fatty acids are of particularly relevant to breast tumors that grow in adipocyte-rich environments. CPT1A is shown to be a potential new target in anti-breast cancer treatment. Modulation of CPT1A expression or activity has been shown to suppress cancer progression. For example, inhibition of CPT1A by pharmacological inhibitor caused severe cytotoxicity and remarkably attenuated beta-oxidation and c-myc-mediated lymphomagenesis in Burkitt's lymphoma. TROLL-8 direct interacts with CPT1A and regulates its activity in committing long-chain fatty acids to catabolic oxidation. However, TROLL-8 interaction does not affect CPT1A expression.

Acetyl-CoA is the end-product of mitochondrial fuel oxidation pathways and therefore, mitochondrial protein acetylation can serve as a convergence point for mitochondrial fuel oxidation pathways. TROLL-8 depletion promoted the cellular acetyl-CoA level and metabolic protein acetylation level. Excitingly, CPT1A displayed the highest acetylation fold change. The hyperacetylation of CPT1A is caused by increased interaction with the acetyltransferase ACAT1, detected by co-IP and LC-MS proteomics analysis and confirmed by WB. Moreover, TROLL-8 silencing induces significant affinity change between CPT1A and metabolic proteins. TROLL-8 modulates CPT1A activity and acetylation through regulating the physical interaction between CPT1A and ACAT1. TROLL-8 silencing induced acetylation can cause allosteric inhibition of CPT1A or block substrate access to CPT1A, leading to reduced CPT1A activity in committing long-chain fatty acyl-CoA to catabolic oxidation. Complementarily, expression of WT CPT1A form, but not the hyperacetylated form, restores TROLL-8 knockdown induced mitochondrial respiration damage and impaired tumorigenesis in breast cancer cells.

We have identified a previously unrecognized lncRNA TROLL-8, which was regulated by the tumor suppressive p53 family transcription factor, TAp63, regulates lipid metabolism through targeting FAO pathway component enzyme, CPT1A. CPT1A has been well-known for its important role in FAO, and inhibition of CPT1A is regarded as an effective therapeutic target in breast cancer. Our findings reveal that lncRNA TROLL-8 interacts with CPT1A and regulates its activity through modulating its interaction with acetyltransferase ACAT1 and involves in regulating mitochondrial respiration and breast cancer apoptosis, giving a new insight into the crosstalk between cellular metabolism and breast cancer progression regulated by TAp63 at the regulatory RNA level.

### c) Methods and Materials

### (1) Cell Culture

MCF10A, DCIS, and CA1D cells were purchased from the Karmanos Cancer Institute (Detroit, MI) and grown in Dulbecco's modified Eagle's medium (DMEM)/F12 (1:1) supplemented with 5% horse serum, 20 ng/ml human epidermal growth factor, 10 ug/ml insulin, and 500 ng/ml hydrocortisone. All cultured cells were maintained at 37° C. and 5% CO₂ and regularly tested mycoplasma negative.

### (2) Gene Expression and Kaplan-Meier Curves of TCGA Data

Our cross-species analysis of coding and non-coding RNAs using RNA sequencing has identified lncRNA TROLL-8 with GENCODE V14 transcript annotation AL161668.12 against a combined reference comprised of Gencode and two lncRNA catalogues. To assess the clinical significance of TROLL-8 lncRNA in breast cancer, we first downloaded the TCGA isoform expression dataset transcriptome profiles from the broad institute and clinical dataset transcriptome profiles from cBioPortal. Then the patient samples were divided into two cohorts according to the median expression of TROLL-8 (high vs. low expression). The expression class was defined as following: expression value=0 was excluded, 0<expression value<=median (of each disease dataset) was defined as low expression, expression value>median (of each disease dataset) was defined as high expression. TROLL-8 expression in breast cancer molecular subtypes was assessed using the subtype data from the brca_tcga_pan_can_atlas_2018.tar.gz data sheet and p-values were calculated using Kruskal-Wallis H-test.

### (3) Gene Expression Analysis by Quantitative Real Time PCR

Total RNA from cell lines was prepared using TRIzol reagent and miRNeasy Mini kit and complementary DNA (cDNA) was synthesized from 5 ug of total RNA using SuperScript II First-Strand Synthesis Kit (Invitrogen) according to the manufacturer's protocol. cDNA was amplified by qRT-PCR using the TaqMan Universal PCR Master Mix (Applied Biosystems) in the QuantStudio 6 flex PCR machine (Applied Biosystems). The RNA expression was normalized to endogenous housekeeping gene human RNA Polymerase II Subunit A (POLR2A) and the relative expression was calculated using 2^{-ΔΔCt} method. Gene-specific primer sequences are listed in Table 1.

(4) Plasmids, siRNAs and shRNAs

pBlueScript II SK (+) TROLL-8 (AL161668.12; Lncipedia Transcript ID: lnc-RNASE13-1:1) was generated by assembling the synthesized TROLL-8 sequence into the pBlueScript II SK (+) phagemid (Agilent Technologies), flanking by the KpnI and SacII sites. For siRNA transient transfection, double-stranded non-coding RNA molecules (50 nM) were transfected using Lipofectamine RNAiMAX (Invitrogen) according to the manufacturer's instructions. The negative siRNA control (siNT) was purchased from Sigma-Aldrich (SIC001-10 NMOL). The siRNA pools used to target the lncRNA TROLL-8 are siTROLL-8 (Sense, 5′-CAUCCAUAAAGAAGGCAUA-3′ (SEQ ID NO: 2), 5′-CCACUUAUUGGCCCUCAUU-3′ (SEQ ID NO: 3), 5′-GACUUGUUCUGUCGCUUCU-3′ (SEQ ID NO: 4). The siRNA targeting ACAT1 was: siACAT1 (SASI_Hs01_00067794). The shRNA targeting CPT1A 3′UTR was encoded by an oligonucleotide with sense-loop-antisense sequence (5′-GGCCGTGATGGTCAGATAATTGGATCCAATTATCTGACCATCACGGCC-3′) (SEQ ID NO: 5) and cloned into pLV-mU6-EF1a-Bsd lentiviral vector (SORT-B24, Biosettia). The negative shRNA control (shNT) was provided by the manufacturer. The pLVpuro-EF1a-CPT1A plasmid was generated by subcloning CPT1A from pcDNA3.1+/C-CPT1A-(K) DYK (NM_001876.3, Genscript) into pLV-EF1a-GFP-Puro (Biosettia). The acetylation mutants pLVpuro-EF1a-CPT1A(K148Q) and pLVpuro-EF1a-CPT1A(K148R) were generated by subcloning CPT1A(K148Q) and CPT1A(K148R) from pcDNA3.1+/C-CPT1A(K148Q)-(K) DYK or pcDNA3.1+/C-CPT1A(K148R)-(K) DYK (Genscript) into pLV-EF1a-GFP-Puro (Biosettia). The generated shRNA and overexpression constructs were then utilized to infect CA1D cells with virus-containing media supplemented with 1000× polybrene for 24 h. Cells were then selected for 4 days with 5 ug/ml blasticidin (shRNA selection) or 1 ug/ml puromycin and collected for the downstream characterization (General Protocol for Lentiviral Transduction, Biosettia).

### (5) ProtoArray Hybridization and Protein Microarray Analysis

In vitro transcription of the sense and antisense TROLL-8 RNAs was performed from the pBluescript II SK (+)—TROLL-8 vector using T7 and T3 polymerase (MEGAscript™ T7/T3 Transcript Kits, ThermoFisher Scientific) in accordance with the manufacturer's instructions. End labeling of in vitro transcribed lncRNA TROLL-8 sense and antisense strands was performed using the Label IT uArray Cy5 labelling kit (Minus) with a labelling efficiency of 3 pmol Cy5 dye per ug RNA following the manufacturer's instructions. 10 pmol of either purified labeled sense or antisense strands (negative control) of TROLL-8 were independently hybridized with recombinant human proteins spotted on the Human Protein Microarrays v5.0 (Invitrogen) slides in buffer containing the following reagents: 40 mM Tris-HCl, pH 8.0, 150 mM sodium chloride, 0.5 mM magnesium acetate, 10 ug/ml Yeast transfer RNA, 10 ug/ml heparin, 1 mM DTT, 0.01% Igepal CA-630, 5% glycerol and 0.2 U/ul RNaseOUT (Invitrogen). Three independent hybridizations for each strand were performed and incubated in the dark at 4° C. for 1 h. After extensive washes, the slides were spin dried and scanned at 635 nm (Cy5) with the GenePix 4000B Microarray scanner (Molecular Devices). GenePix Pro 6.1 software (Molecular Devices) was used to determine the intensity of the green-fluorescent signal (635 nm) at each protein spot location. Binding signal intensities were reflected by the ratio of the intensity of the 635 nm signal (F635) divided by the local background intensity (B635) at each of the protein spot. Quantification of lncRNA-protein interactions were determined by the mean signal intensities of the duplicate spots for a given protein. Signal-above-background method was used to filter data. Proteins with mean signal intensities greater than two (F635/B635>2) were filtered into the analysis to run through the Ingenuity Pathway Analysis (IPA) (QIAGEN IPA) to screen for pathways and biological processes.

(6) In Vitro lncRNA Pull-Down Coupled with Subsequent Protein Detection

For in vitro RNA pull-down, the in vitro transcribed lncRNA were end-labelled with desthiobiotin (magnetic RNA-protein pull-down kit, Pierce) according to the manufacturer's instructions. 50 pmol of biotin-labelled lncRNA was pre-incubated with 50 ul streptavidin magnetic beads for 30 min at RT with gentle agitation. Magnetic stand was used to collect streptavidin magnetic bead-bound lncRNA. The bead-lncRNA complex was then incubated with cell lysate of CA1D cells overexpressing FLAG-tagged CPT1A (pLV-EF1a-CPT1A-puro, Biosettia) overnight at 4° C. with gentle end-to-end rotation. Beads were washed with 1× Wash Buffer and resuspended in 50 ul of Elution Buffer provided in the kit. The eluted RNA-bound proteins were separated by SDS-PAGE and detected with anti-CPT1A monoclonal antibody (ab128568, Abcam, 1:1000).

### (7) Western Blot Analysis

50 ug to 300 ug of protein were electrophoresed on a 10% SDS-PAGE and transferred to nitrocellulose membrane as described before. Membrane was then immunoblotted with anti-CPT1A (ab128568, Abcam, 1:1000), anti-ACAT1 (ab168342, 1:1000, Abcam), anti-acetylated-lysine (#9441, 1:1000, Cell Signaling), anti-alpha-tubulin (ab52866, 1:1000, Abcam), anti-VDAC (ab154856, 1:1000, Abcam), anti-beta-actin (A5441, 1:10,000, Sigma-Aldrich) at 4° C. for 18 h and then immunoblotted with appropriate secondary antibodies conjugated to horseradish peroxidase (1:5,000) (Jackson Lab) by incubation for 1 h at room temperature. Beta-actin was served as the loading control. Signal detection was performed using ECL Prime Western Blotting Detection Reagent (Amersham) according to the manufacturer's protocol and LI-COR infrared (IR) laser-based quantitation.

### (8) Co-Immunoprecipitation (CoIP) Assay

4 mg cell lysate of CA1D cells were prepared using RIPA buffer and used to test the interaction between CPT1A and ACAT1. 4 mg cell lysate of either CA1D cells transfected with siRNAs for TROLL-8, or with the non-targeting siRNA as a negative control, were used to test the change in the interaction between CPT1A and ACAT1. The CoIP assay was performed according to the protocol provided with the anti-CPT1A primary antibody (15184-1-AP). Add anti-CPT1A primary antibody (15184-1-AP, Proteintech) or negative control normal rabbit IgG (#2729, Cell Signaling) corresponding to the primary antibody source to the lysate with gentle rocking at 4° C. for 12 h. 50 ul Protein G Dynabeads slurry were added to capture the immunocomplex with gentle rocking at 4° C. for 12 h. Collect the beads with magnetic stand and discard the supernatant. Wash the beads 3 times with 1 ml 0.2% TBST, collect the beads with magnetic stand and discard the supernatant. Elute the pellet twice with 40 ul 0.10M Glycine, 0.05M Tris-HCl (pH 2.5) elution buffer containing 500 mM NaCl. Pool elutions and neutralize by 10 ul Alkali neutralization buffer (1M NaOH). Add 30 ul 4×SDS sample buffer to a final of 1× elution. Heat the elution at 95° C. for 5 min and collect the supernatant with magnetic stand and discard the beads. The interaction was then detected via western blot using the following primary antibodies: CPT1A (ab128568, Abcam), ACAT1 (ab168342, Abcam), and beta-actin (A5441, Sigma-Aldrich).

### (9) In Situ Hybridization of Human Tissue Microarrays

TMA of breast cancer progression (BR480a, US Biomax) and TMA of breast normal adjacent tissue and cancer tissue (BRN801c, US Biomax) were used for the ISH assay. The 5′ and 3′ digoxigenin (DIG) labelled LNA probes (Qiagen) utilized for ISH were: TROLL-8 (5′-TACAGAGGCAAGCGGTGAACT-3′) (SEQ ID NO: 6) and the detection control probe (339508, Qiagen, 5′-GTGTAACACGTCTATACGCCCA-3′)(SEQ ID NO: 7). The ISH was performed using the Qiagen miRCURY LNA miRNA ISH optimization kit for FFPE tissues according to the manufacturer's protocols. Briefly, 200 nM of the double DIG labelled LNA probes were hybridized to the TMA slides and incubated at 55° C. for 1 h in the Dako hybridizer (Agilent). Alkaline phosphate (AP)-conjugated anti-DIG antibody Fag fragment (11093274910, Sigma-Aldrich, 1:400) was added to detect the LNA probes. This step is followed by AP substrate, NBT-BCIP (11697471001, Roche) development and counterstaining with Nuclear Fast Red™ (H-3403, Vector Laboratories). The LNA probe binding was visualized by a chromogenic conversion of water soluble NBT and BCIP substrates into a water- and alcohol-insoluble, dark-blue NBT-BCIP precipitate. The signal intensity and the percentage of positive staining area were measured. The ISH score was then quantified by multiplying the signal intensity by the percentage of positive staining area.

### (10) Seahorse Assay

The oxygen consumption rate (OCR) of cells was measured on the Seahorse XF96 extracellular flux analyzer (Agilent Technologies) using a Seahorse XF Cell Mito Stress Test kit in accordance with the manufacturer's instructions. Briefly, siNT or siTROLL-8 CA1D cells (3.0×10⁴ cells/well) were seeded in 6 wells (technical replicates) of a 96 well Agilent Seahorse XF Cell Culture Microplate in full growth medium and attached overnight. Hydrate a sensor cartridge in Seahorse XF Calibrant in a non-CO₂ incubator at 37° C. overnight. On the day of assay, cells were carefully washed and the growth medium was replaced with prewarmed Seahorse XF base medium (Agilent Technologies) supplemented with 5 mM HEPES, 10 mM glucose, 1 mM sodium pyruvate, and 2 mM L-Glutamine, pH 7.4). The plates of cells in assay medium were incubated in a non-CO₂ incubator at 37° C. for 1 h. Prepare compound stock solutions for loading sensor cartridge ports and load the XF96 sensor cartridge to Seahorse XF96 analyzer. Take three basal measurements and determine oxygen and proton concentration in the medium. The ATP synthase inhibitor oligomycin (final well concentration 1 μM), FCCP (final well concentration 1 μM), and Rot/AA (final well concentration 0.5 μM) were sequentially added for three further measurements of OCR by inhibiting ATP production, stimulating oxygen consumption to reach the maximum, and shut down mitochondrial respiration, respectively. For the mitochondrial glucose and glutamine supplementation assays, the assay medium was prepared by supplementing the prewarmed Seahorse XF base medium with 5 mM HEPES and 10 mM glucose or 5 mM HEPES and 2 mM glutamine. For the fatty acid supplementation assay, the assay medium was prepared by supplementing 1×KHB buffer (111 mM NaCl, 4.7 mM KCl, 1.25 mM CaCl₂, 2.0 mM MgSO4, and 1.2 mM NaH₂PO4) with 2.5 mM glucose, 0.5 mM carnitine and 5 mM HEPES. For the mitochondrial fuel pathway inhibitor assays, the pathway inhibitors were prepared as glutamine oxidation inhibitor BPTES (final well concentration 3.0 μM), fatty acid oxidation inhibitor Etomoxir (final well concentration 4.0 μM) and glucose oxidation inhibitor UK5099 (final well concentration 2.0 μM) and loaded to port A of the sensor cartridge. Oligomycin, FCCP, and Rot/AA were loaded to port B, C, and D of the sensor cartridge and assays were performed using the Seahorse XF Cell Mito Stress Test protocol.

### (11) Cell-Fractionation and Mitochondrial Isolation

Mitochondrial and cytoplasmic extracts were prepared from CA1D cells with Mitochondrial Isolation Kit for Cultured Cells (Thermo Scientific) according to manufacturer's instructions. Briefly, 2×10⁷ cells were collected by centrifugation, washed once in ice-cold PBS and resuspended in mitochondrial isolation reagent A with vortexing at medium speed. The cell suspension was then lysed using Dounce Tissue Grinder and mixed with additional reagent A and reagent C. The cell suspension mixture was centrifuged (4° C., 700×g, 5 min) and supernatant was collected and centrifuged again at 3000×g for 15 mins. Supernatant was set aside and treated as the cytoplasmic fraction. The pellet contained the isolated mitochondria and was washed with reagent C (centrifugation at 12,000×g for 5 min). The isolated mitochondria were lysed with 2% CHAPS in Tris-buffered saline (TBS, 25 mM Tris, 0.15M NaCl, pH 7.2) and protein concentration was measured using Bradford Assay.

### (12) Analysis by Liquid Chromatography-Mass Spectrometry (LC-MS/MS)

1.5×10⁶ CA1D cells were transfected with either siNT (negative control) or siTROLL-8. 48 h after the transfection, the cells were lysed. Cell lysates were applied to run SDS-PAGE gel or do CoIP assay. The SDS-PAGE gel bands or the immunoprecipitated proteins were extracted, digested with trypsin, and analyzed via LC-MS/MS and the identified peptides are listed in Supplementary Data 1. For tandem mass spectrometry peptide sequencing experiments, a nanoflow ultra-high performance liquid chromatography (UHPLC) (RSLC, Dionex, Sunnyvale, CA) coupled to an electrospray bench top orbitrap mass spectrometer (Q-Exactive plus, Thermo, San Jose, CA) was used. The samples were first loaded onto a pre-column (2 cm×100 μm ID packed with C18 reversed-phase resin, 5 μm, 100 Å) and washed for 8 mins with aqueous 2% acetonitrile and 0.04% trifluoroacetic acid. The trapped peptides were eluted onto the analytic column (C18, 75 μm ID×50 cm, 2 μm, 100 Å, Dionex, Sunnyvale, CA) followed by a 120-minute gradient with 95% solvent A (2% acetonitrile+0.1% formic acid) for 8 mins, solvent B (90% acetonitrile+0.1% formic acid) from 5% to 38.5% for 90 mins, then solvent B from 50% to 90% B for 7 mins and held at 90% for 5 mins, followed by solvent B from 90% to 5% in 1 min and re-equilibration for 10 mins. The flow rate on analytic column was 300 nl/min. 16 tandem mass spectra were collected in a data-dependent manner following each survey scan. Both MS and MS/MS scans were performed in Orbitrap to obtain accurate mass measurement using 60 second exclusion for previously sampled peptide peaks. Sequest and Mascot searches were performed against the Swiss-Prot human database downloaded on Jun. 12, 2016. Two trypsin missed cleaves were allowed, the precursor mass tolerance was 20 ppm. MS/MS mass tolerance was 0.05 Da. Dynamic modifications included carbamidomethylation (Cys), oxidation (Met) and acetylation (Lys).

### (13) Analysis by Liquid Chromatography-Mass Spectrometry (LC-MS)

1.5×10⁶ CA1D cells were transfected with either siNT (negative control) or siTROLL-8. 48 h after the transfection, the cells were collected and washed 2-3 times with ice-cold PBS. To analyze global metabolite abundances regulated by TROLL-8, an aliquot of the internal standard mixture was added into siNT and siTROLL-8 cell samples for untargeted metabolomics analysis. The internal standards were obtained from Cambridge Isotope Labs and include the following labelled compounds: Glucose (2,3,4,5,6-¹³C5), D-Glucose-6-phosphate (U-¹³C6), D-Fructose-1,6-bisphosphate (U-¹³C6), L-Serine (¹³C3), Glycine (1,2-¹³C2), L-Cysteine (3,3-D2), Phosphoenol Pyruvate (2,3-¹³C2), Lactate (3,3,3-D3), Pyruvate (D3), Acetyl-1,2-¹³C2 CoA, Citric Acid (2,2,4,4-D4), Alpha-Ketoglutaric Acid (1,2,3,4-¹³C4), Succinic Acid (D4), Fumaric Acid (D4), DL-Malic Acid (2,3,3-D3), D-Fructose-6-phosphate (U-¹³C6). 1 ml of precooled 80% methanol extraction solvent (kept in the −80° C. freezer at least 1 h prior to extraction) was added to the sample for protein precipitation. After addition of the extraction solvent, the samples were vortexed and centrifuged at 18,800×g (Microfuge 22R, Beckman Coulter) at 0° C. for 10 min. Then, the samples were incubated for 30 min in a −80° C. freezer to increase metabolite extraction. After incubation, the samples were immediately centrifuged at 18,800×g at 4° C. for 10 min. Then the supernatant was transferred to a new microcentrifuge tube for drying in SpeedVac vacuum concentrator (Thermo Fisher Scientific). The protein pellet was resolubilized using aqueous 20 mM HEPES with 8M urea for Bradford Assay to measure the protein concentration. Dried metabolites were re-dissolved in 20 μl aqueous 80% methanol. Ultra-high performance liquid chromatography-high resolution mass spectrometry (UHPLC-HRMS) was performed using a Vanquish UHPLC interfaced with a Q Exactive HF quadrupole-orbital ion trap mass spectrometer (Thermo, San Jose, CA). Chromatographic separation was performed using a SeQuant ZIC-pHILIC guard column (2.1 mm ID×20 mm length, 5 μm particle size) and a SeQuant ZIC-pHILIC LC column (2.1 mm ID×150 mm length, 5 μm particle size, MilliporeSigma, Burlington, MA). Mobile phase A was aqueous 10 mM ammonium carbonate and 0.05% ammonium hydroxide, and mobile phase B was 100% acetonitrile. The gradient program included the following steps: start at 80% B, a linear gradient from 80 to 20% B over 13 min, stay at 20% B for 2 min, return to 80% B in 0.1 min, and re-equilibration for 4.9 min for a total run time of 20 min. The flow rate was set to 0.250 ml/min. The autosampler was cooled to 5° C. and the column temperature was set to 30° C. Sample injection volume was 2 μl for both positive ion mode and negative ion mode electrospray ionization. Full MS was performed in positive and negative mode separately detecting ions from m/z 65 to m/z 900. MZmine software, version 3.39, was used to identify and quantify metabolites by matching by m/z and RT to an in-house library. Data normalization was carried out using the protein concentration. For acetyl-CoA detection, spike heavily labelled internal standards (3 labelled Acyl CoAs) into each sample. For fatty acyl-carnitine detection, spike heavily labelled internal standards (3 labelled Acyl Carnitine) into each sample. An aliquot (300 μl) of cold 80% methanol extraction solvent (leave in −80° C. at least one hour prior to the experiments) is added to the sample for protein precipitation. After vortexing, the samples are then incubated in the −80° C. freezer for 30 mins, followed by centrifugation at 18,800×g (Microfuge 22R, Beckman Coulter) at 0° C. for 10 mins. Then the supernatant is transferred to new Eppendorf tubes for drying in SpeedVac vacuum concentrator (Thermo Fisher Scientific). The protein pellet is left behind and proceeds for protein concentration measurement by Bradford Assay. The dried pellet is re-dissolved in 10 ul 80% methanol for the following UHPLC-MS analysis for acetyl-CoA detection, which was performed using a Vanquish LC (Thermo, San Jose, CA) interfaced with a Q Exactive HF mass spectrometer (Thermo, San Jose, CA). Chromatographic separation was performed on a AccureCore Vanquish C18+ (2.1 mm×100 mm, 1.5 μm particle size, Thermo, San Jose, CA). The mobile phase A was 10:90 ACN:H2O with 15 mM NH4OH, and the mobile phase B was 100% acetonitrile. The total running time is 15 min. The column temperature was set to 30° C., and the injection volume is 2 μl. The Parallel Reaction Monitoring (PRM) is performed in positive mode and the isolation window is 3.0 m/z with 0.5 m/z offset. Xcalibur was used for the data analysis. For fatty acyl-carnitine detection, UHPLC-MS was performed using a Vanquish LC (Thermo, San Jose, CA) interfaced with a Q Exactive FOCUS mass spectrometer (Thermo, San Jose, CA). Chromatographic separation was performed on a ACQUITY UPLC BEH Amide column (2.1 mm×150 mm, 1.7 μm particle size, Waters, Milford, MA). The mobile phase A was 10 mM ammonium carbonate and 0.05% ammonium hydroxide in water, and the mobile phase B was 100% acetonitrile. The total running time is 15 min. The column temperature was set to 30° C., and the injection volume is 2 ul. The full MS is performed in positive and the mass scan range is 150 to 500 m/z. Skyline was used for the data analysis.

### (14) Palmitate, Glucose, and Glutamine Labelling and Tracing Experiments

To assess the FAO pathway, 1.5×10⁶ CA1D cells were transfected with siNT (negative control) or siTROLL-8 and grown in DMEM/F-12 growth medium containing 200 μM uniformly ¹³C labelled (U-¹³C) palmitate (Cambridge Isotope Laboratories, CLM-409-0.5) and 5% delipidated FBS (Gemini Bio-products, 900-123). For U-¹³C glucose and U-¹³C glutamine tracing experiments, cells were transfected and grown in DMEM/F-12 growth medium containing 12 mM uniformly ¹³C labelled (U-¹³C) glucose (Cambridge Isotope Laboratories, CLM-481-0.5) or 2 mM uniformly ¹³C labelled (U-¹³C) glutamine (Cambridge Isotope Laboratories, CLM-1822-H-0.5). After labelling for 48 h, cells were harvested, extracted, and analyzed as described in the LC-MS metabolite abundance test protocol. All processes were carried out on ice. No internal standards were added into the isotope tracer samples. Data normalization was carried out using the protein concentration. EI-MAVEN was used for the data analysis.

### (15) Statistical Analysis

Statistical data analysis was performed using GraphPad Prism 9 on experiments of at least three independent replicates. Details of data collection and statistical tests are discussed in the figure legends. Western blot images are representative of three independent experiments giving similar results.

### D. SEQUENCES

SEQ ID NO: 1
Tap63 Binding site for TROLL-8 lncRNA
GAACATGATCGATCCATGTCA
SEQ ID NO: 2
siRNA specific for TROLL-8 _{(AL161668.12}
CATCCATAAAGAAGGCATA
SEQ ID NO: 3
siRNA specific for TROLL-8 _{(AL161668.12}
CCACTTATTGGCCCTCATT
SEQ ID NO: 4
siRNA specific for TROLL-8 _{(AL161668.12}
GACTTGTTCTGTCGCTTCT
SEQ ID NO: 5
shRNA targeting CPT1A
GGCCGTGATGGTCAGATAATTGGATCCAATTATCTGACCATCACGGCC
SEQ ID NO: 6
digoxigenin (DIG) labelled LNA probes
TACAGAGGCAAGCGGTGAACT
SEQ ID NO: 7
digoxigenin (DIG) labelled LNA probes
GTGTAACACGTCTATACGCCCA

### E. REFERENCES

Altman B J, Stine Z E, Dang C V. 2016. Nat Rev Cancer 16: 749
Anderson K A, Hirschey M D. 2012. Essays Biochem 52: 23-35
Antonenkov V D, Croes K, Waelkens E, Van Veldhoven P P, Mannaerts G P. 2000. Eur J Biochem 267: 2981-90
Avanzato D, Pupo E, Ducano N, Isella C, Bertalot G, et al. 2018. Cancer Res 78: 3432-44
Bhan A, Soleimani M, Mandal S S. 2017. Cancer Res 77: 3965-81
Brenton J D, Carey L A, Ahmed A A, Caldas C. 2005. J Clin Oncol 23: 7350-60
Burgess E A, Sylven B. 1962. Cancer Res 22: 581-8
Cabili M N, Trapnell C, Goff L, Koziol M, Tazon-Vega B, et al. 2011. Genes Dev 25: 1915-27
Cairns R A, Harris I S, Mak T W. 2011. Nat Rev Cancer 11: 85-95
Camarda R, Zhou A Y, Kohnz R A, Balakrishnan S, Mahieu C, et al. 2016. Nat Med 22: 427-32
Carmeliet P. Jain R K. 2000. Nature 407: 249-57
Carracedo A, Weiss D, Leliaert A K, Bhasin M, de Boer V C, et al. 2012. J Clin Invest 122: 3088-100
Chen R, Liu Y, Zhuang H, Yang B, Hei K, et al. 2017. Nucleic Acids Res 45: 9947-59
Choudhary C, Kumar C, Gnad F, Nielsen M L, Rehman M, et al. 2009. Science 325: 834-40
Cimen H, Han M J, Yang Y, Tong Q, Koc H, Koc E C. 2010. Biochemistry 49: 304-11
de Souza C O, Kurauti M A, Silva Fde F, de Morais H, Curi R, et al. 2015. Cell Physiol Biochem 36: 1659-69
Doyen J, Trastour C, Ettore F, Peyrottes I, Toussant N, et al. 2014. Biochem Biophys Res Commun 451: 54-61
Eng J K, McCormack A L, Yates J R. 1994. J Am Soc Mass Spectrom 5: 976-89
Fan J, Lin R, Xia S, Chen D, Elf S E, et al. 2016. Mol Cell 64: 859-74
Fan T W, Lane A N, Higashi R M, Farag M A, Gao H, et al. 2009. Mol Cancer 8: 41
Feng J, Bi C, Clark B S, Mady R, Shah P, Kohtz J D. 2006. Genes Dev 20: 1470-84
Fukao T, Song X Q, Mitchell G A, Yamaguchi S, Sukegawa K, et al. 1997. Pediatr Res 42: 498-502
Fukao T, Yamaguchi S, Nagasawa H, Kano M, Orii T, et al. 1990. J Inherit Metab Dis 13: 757-60
Garrido P, Osorio F G, Moran J, Cabello E, Alonso A, et al. 2015. J Cell Physiol 230: 191-8
Gatenby R A, Smallbone K, Maini P K, Rose F, Averill J, et al. 2007. Br J Cancer 97: 646-53
Gatza M L, Silva G O, Parker J S, Fan C, Perou C M. 2014. Nat Genet 46: 1051-9
German N J, Yoon H, Yusuf R Z, Murphy J P, Finley L W, et al. 2016. Mol Cell 63: 1006-20
Glozak M A, Sengupta N, Zhang X, Seto E. 2005. Gene 363: 15-23
Goldhirsch A, Wood W C, Coates A S, Gelber R D, Thurlimann B, et al. 2011. Ann Oncol 22: 1736-47
Han J, Qu H, Han M, Ding Y, Xie M, et al. 2021. Oncogene 40: 833-47
Harrow J, Frankish A, Gonzalez J M, Tapanari E, Diekhans M, et al. 2012. Genome Res 22: 1760-74
He J, Li F, Zhou Y, Hou X, Liu S, et al. 2020. Cancer Let 469: 419-28
He Z, You C, Zhao D. 2018. Biochem Biophys Res Commun 500: 569-76
Helmlinger G, Yuan F, Dellian M, Jain R K. 1997. Nat Med 3: 177-82
Hu J, Shan Y, Ma J, Pan Y, Zhou H, et al. 2019. Int J Cancer 145: 450-60
Huang N S, Chi Y Y, Xue J Y, Liu M Y, Huang S, et al. 2016. Oncotarget 7: 37957-65
Huarte M, Guttman M, Feldser D, Garber M, Koziol M J, et al. 2010. Cell 142: 409-19
Iriondo O, Rabano M, Domenici G, Carlevaris O, Lopez-Ruiz J A, et al. 2015. Oncotarget 6: 31721-39
Jin L, Alesi G N, Kang S. 2016. Oncogene 35: 3619-25
Keung W, Ussher J R, Jaswal J S, Raubenheimer M, Lam V H, et al. 2013. Diabetes 62: 711-20
Kim J E, Chen J, Lou Z. 2008. Nature 451: 583-6
Kim S C, Sprung R, Chen Y, Xu Y, Ball H, et al. 2006. Mol Cell 23: 607-18
Korman S H. 2006. Mol Genet Metab 89: 289-99
Kruszynska Y T, Sherratt H S. 1987. Biochem Pharmacol 36: 3917-21
Lampa M, Arlt H, He T, Ospina B, Reeves J, et al. 2017. PLoS One 12: e0185092
Li B, Chen P, Qu J, Shi L, Zhuang W, et al. 2014. J Biol Chem 289: 29365-75
Li D, Liu X, Zhou J, Hu J, Zhang D, et al. 2017. Hepatology 65: 1612-27
Li H J, Sun X M, Li Z K, Yin Q W, Pang H, et al. 2017. Cell Physiol Biochem 43: 2548-61
Li J, Zhao S, Zhou X, Zhang T, Zhao L, et al. 2013. PLoS One 8: e74623
Li S, Zhou T, Li C, Dai Z, Che D, et al. 2014. PLoS One 9: e97330
Li Y, Prives C. 2007. Oncogene 26: 2220-5
Liedtke C, Mazouni C, Hess K R, Andre F, Tordai A, et al. 2008. J Clin Oncol 26: 1275-81
Lin W, Zhou Q, Wang C Q, Zhu L, Bi C, et al. 2020. Int J Biol Sci 16: 1194-206
Liu Y W, Xia R, Lu K, Xie M, Yang F, et al. 2017. Mol Cancer 16: 39
Longo N, Amat di San Filippo C, Pasquali M. 2006. Am J Med Genet C Semin Med Genet 142C: 77-85
Louie S M, Grossman E A, Crawford L A, Ding L, Camarda R, et al. 2016. Cell Chem Biol 23: 567-78
Maher E A, Marin-Valencia I, Bachoo R M, Mashimo T, Raisanen J, et al. 2012. NMR Biomed 25: 1234-44
Manabe Y, Toda S, Miyazaki K, Sugihara H. 2003. J Pathol 201: 221-8
Martinez-Reyes I, Cardona L R, Kong H, Vasan K, McElroy G S, et al. 2020. Nature 585: 288-92
McGarry J D, Brown N F. 1997. Eur J Biochem 244: 1-14
Menzies K J, Zhang H, Katsyuba E, Auwerx J. 2016. Nat Rev Endocrinol 12: 43-60
Michishita E, McCord R A, Berber E, Kioi M, Padilla-Nash H, et al. 2008. Nature 452: 492-6
Mongelli A, Martelli F, Farsetti A, Gaetano C. 2019. Front Physiol 10: 369
Morais-Santos F, Granja S, Miranda-Goncalves V, Moreira A H, Queiros S, et al. 2015. Oncotarget 6: 19177-89
Morris K V, Mattick J S. 2014. Nat Rev Genet 15: 423-37
Murthy M S, Pande S V. 1987. Proc Nat Acad Sci USA 84: 378-82
Nagano T, Mitchell J A, Sanz L A, Pauler F M, Ferguson-Smith A C, et al. 2008. Science 322: 1717-20
Nagarajan A, Malvi P, Wajapeyee N. 2016. Trends Cancer 2: 365-77
Napoli M, Li X, Ackerman H D, Deshpande A A, Barannikov I, et al. 2020. Nat Commun 11:
Pacilli A, Calienni M, Margarucci S, D'Apolito M, Petillo O, et al. 2013. J Nati Cancer Inst 105: 489-98
Palaskas N, Larson S M, Schultz N, Komisopoulou E, Wong J, et al. 2011. Cancer Res 71: 5164-74
Park J H, Vithayathil S, Kumar S, Sung P L, Dobrolecki L E, et al. 2016. Cell Rep 14: 2154-65
Perkins D N, Pappin D J, Creasy D M, Cottrell J S. 1999. Electrophoresis 20: 3551-67
Pucci S, Zonetti M J, Fisco T, Polidoro C, Bocchinfuso G, et al. 2016. Oncotarget 7: 19982-96
Puzio-Kuter A M. 2011. Genes Cancer 2: 385-91
Qiu X, Brown K, Hirschey M D, Verdin E, Chen D. 2010. Cell Metab 12: 662-7
Quehenberger O, Armando A M, Dennis E A. 2011. Biochim Biophys Acta 1811: 648-56
Rinn J L, Chang H Y. 2012. Annu Rev Biochem 81: 145-66
Rios Garcia M, Steinbauer B, Srivastava K, Singhal M, Mattijssen F, et al. 2017. Cell Metab 26: 842-55 e5
Robertson-Tessi M, Gillies R J, Gatenby R A, Anderson A R. 2015. Cancer Res 75: 1567-79
Schlaepfer I R, Nambiar D K, Ramteke A, Kumar R, Dhar D, et al. 2015. Oncotarget 6: 22836-56
Schmitt A M, Chang H Y. 2016. Cancer Cell 29: 452-63
Schmitt A M, Chang H Y. 2017. Cold Spring Harb Perspect Med 7
Seo J S, Diloknawarit P, Park B S, Chua N H. 2019. New Phytol 221: 2067-79
Shen L, O'Shea J M, Kaadige M R, Cunha S, Wilde B R, et al. 2015. Proc Natl Acad Sci USA 112: 5425-30
Shibue T, Weinberg R A. 2017. Nat Rev Clin Oncol 14: 611-29
Sorlie T, Perou C M, Tibshirani R, Aas T, Geisler S, et al. 2001. Proc Natl Acad Sci USA 98: 10869-74
Sorlie T, Tibshirani R, Parker J, Hastie T, Marron J S, et al. 2003. Proc Nat Acad Sci USA 100: 8418-23
Sotiriou C, Neo S Y, McShane L M, Korn E L, Long P M, et al. 2003. Proc Natl Acad Sci USA 100:10393-8
Su X, Chakravarti D, Cho M S, Liu L, Gi Y J, et al. 2010. Nature 467: 986-90
Su X, Gi Y J, Chakravarti D, Chan I L, Zhang A, et al. 2012. Cell Metab 16: 511-25
Sullivan M R, Danai L V, Lewis C A, Chan S H, Gui D Y, et al. 2019. Elife 8
Tsai M C, Manor O, Wan Y, Mosammaparast N, Wang J K, et al. 2010. Science 329: 689-93
Tumanov S, Bulusu V, Kamphorst J J. 2015. Methods Enzymol 561: 197-217
Tung S, Shi Y, Wong K, Zhu F, Gorczynski R, et al. 2013. Blood 122: 969-80
Ulitsky I, Shkumatava A, Jan C H, Sive H, Bartel D P. 2011. Cell 147: 1537-50
Vacanti N M, Divakaruni A S, Green C R, Parker S J, Henry R R, et al. 2014. Mol Cell 56: 425-35
Valentino A, Calarco A, Di Salle A, Finicelli M, Crispi S, et al. 2017. Oncogene 36: 6030-40
van Geldermalsen M, Wang Q, Nagarajah R, Marshall A D, Thoeng A, et al. 2016. Oncogene 35: 3201-8
Vander Heiden M G, Cantley L C, Thompson C B. 2009. Science 324: 1029-33
Wang P, Xue Y, Han Y, Lin L, Wu C, et al. 2014. Science 344: 310-3
Wang Z, Jiang Q, Dong C. 2020. Cancer Biol Med 17: 44-59
Ward P S, Thompson C B. 2012. Cancer Cell 21: 297-308
Wu M, An J, Zheng Q, Xin X, Lin Z, et al. 2016. Oncotarget 7: 66525-39
Xiong Y, Guan K L. 2012. J Cell Biol 198: 155-64
Yao C H, Liu G Y, Wang R, Moon S H, Gross R W, Patti G J. 2018. PLoS Biol 16: e2003782
Yoon J H, Abdelmohsen K, Kim J, Yang X, Martindale J L, et al. 2013. Nat Commun 4: 2939
Zaidi N, Lupien L, Kuemmerle N B, Kinlaw W B, Swinnen J V, Smans K. 2013. Prog Lipid Res 52: 585-9
Zhang T, Hu H, Yan G, Wu T, Liu S, et al. 2019. Technol Cancer Res Treat 18: 1533033819843889
Zhang X, Wang W, Zhu W, Dong J, Cheng Y, et al. 2019. Int J Mol Sci 20
Zhao S, Xu W, Jiang W, Yu W, Lin Y, et al. 2010. Science 327: 1000-4
Zhao W, Kruse J P, Tang Y, Jung S Y, Qin J, Gu W. 2008. Nature 451: 587-90
Zhao X, Hu X. 2019. J Cell Physiol 234: 20992-1004

1. A method of assessing tumor grade and/or progression of a cancer and/or metastasis in a subject comprising obtaining a tissue sample from a subject and measuring the expression level of the long non-coding RNA for TROLL-8; wherein the higher the level of lncRNA for TROLL-8, the greater the severity and/or invasiveness of the tumor is indicated.

2. A method of assessing the efficacy of a cancer treatment regimen administered to a subject comprising obtaining a tissue sample from a subject and measuring the expression level of the long non-coding RNA for TROLL-8 relative to a control.

3. The method of assessing the efficacy of a cancer treatment regimen of claim 2; wherein when the expression level of lncRNA for TROLL-8 is i) higher than a negative control, ii) equivalent to or has not decreased relative to a positive control and/or equivalent to or has not decreased relative to a positive control; indicates that the treatment regimen is not efficacious.

4. The method of assessing the efficacy of a cancer treatment regimen of claim 2, wherein the positive control is a reference gene or pretreatment sample from the subject whose cancer treatment regimen is being assessed.

5. A method of detecting the presence of a cancer in a subject comprising obtaining a tissue sample from the subject and assaying the tissue sample for the presence and/or expression level of the long non-coding RNA for TROLL-8; wherein the presence or an increase in lncRNA for TROLL-8, indicates the presence of a cancer in the tissue sample from the subject.

6. A method of treating a cancer in a subject comprising obtaining a tissue sample from a subject receiving a cancer treatment regimen and measuring the expression level of the long non-coding RNA for TROLL-8; wherein when the expression level of lncRNA for TROLL-8 is i) higher than a negative control and/or equivalent to or has not decreased relative to a positive control; indicates that the treatment regimen is not efficacious; and wherein the method further comprises changing the treatment regimen when the treatment regimen is not efficacious.

7. A method of treating a cancer in a subject comprising i) obtaining a tissue sample from the subject; ii) assaying the tissue sample for the presence and/or expression level of the long non-coding RNA for TROLL-8; wherein the presence of lncRNA for TROLL-8 indicates the presence of a cancer in the tissue sample from the subject; and iii) administering to a subject an agent that knocks down expression of TROLL-8 or increases expression of carnitine palmitoyltransferase 1A (CPT1A).

8. The method of treating a cancer in a subject of claim 6, wherein expression of TROLL-8 is knocked down through the administration of one or more RNA-targeted therapeutics.

9. The method of claim 8, wherein the one or more RNA-targeted therapeutics comprises antisense oligonucleotides, siRNA, shRNA, ribozymes, transcription activator-like effector nucleases (TALEN), zinc finger nucleases (ZFNs) and/or clustered regularly interspaced short palindromic repeats/associated (CRISPR/Cas) nucleases.

10. The method of claim 9, wherein the siRNA comprises the sequence as set forth in SEQ ID NO: 2, SEQ ID NO: 3, or SEQ ID NO: 4.

11. The method of treating a cancer in a subject of claim 7, wherein the treatment comprises administering to the subject carnitine palmitoyltransferase 1A (CPT1A) or a vector that overexpresses CPT1A.

12. (canceled)

13. The method of claim 6, wherein the cancer is a breast cancer.

14. The method of claim 13, wherein the cancer is a triple negative breast cancer (TNBC) or invasive ductal carcinoma (IDC).

15. The method of claim 6, wherein the cancer comprises a cancer with a KRAS^{G12C} mutation or p53 mutation.

16. The method of treating a cancer in a subject of claim 7, wherein expression of TROLL-8 is knocked down through the administration of one or more RNA-targeted therapeutics.

17. The method of claim 16, wherein the one or more RNA-targeted therapeutics comprises antisense oligonucleotides, siRNA, shRNA, ribozymes, transcription activator-like effector nucleases (TALEN), zinc finger nucleases (ZFNs) and/or clustered regularly interspaced short palindromic repeats/associated (CRISPR/Cas) nucleases.

18. The method of claim 17, wherein the siRNA comprises the sequence as set forth in SEQ ID NO: 2, SEQ ID NO: 3, or SEQ ID NO: 4.

19. The method of claim 7, wherein the cancer is a breast cancer.

20. The method of claim 19, wherein the cancer is a triple negative breast cancer (TNBC) or invasive ductal carcinoma (IDC)

21. The method of claim 7, wherein the cancer comprises a cancer with a KRAS^{G12C} mutation or p53 mutation.
